(19) United States
(12) Patent Application Publication
(10) Pub. No.: US 2002/0037244 A1
(43) Pub. Date: Mar. 28, 2002

(54) Air cleaner

(76) Inventors: Toshio Takahashi, Nishikasugai-gun (JP); Kazushi Noda, Nishikasugai-gun (JP); Kazuhiro Sakai, Nishikasugai-gun (JP); Takao Mukogawa, Nishikasugai-gun (JP); Shinichi Taniguchi, Toyota-shi (JP)

Correspondence Address: PILLSBURY WINTHROP LLP, 1600 TYSONS BOULEVARD, MCLEAN, VA, 22102, US

(21) Appl. No.: 09/960,968

(22) Filed: Sep. 25, 2001

(30) Foreign Application Priority Data
Sep. 26, 2000 (JP) P. 2000-291989
Sep. 26, 2000 (JP) P. 2000-291991
Sep. 26, 2000 (JP) P. 2000-291994
Sep. 26, 2000 (JP) P. 2000-292009
Sep. 26, 2000 (JP) P. 2000-292015

(51) Int. Cl. A61L 2/00; A61L 9/00

(52) U.S. Cl. 422/297; 422/305; 422/306; 422/906

(57) ABSTRACT

The air cleaner comprises an air cleaner body, an air passage, a fan for forming air flow flowing in the air passage and a filter to which pollutant included in the air. The air cleaner body has fitting parts. When the turned angle of the grille with the body is small, a turning arm of the grille can be inserted or detached into/from the space between the fitting parts when the turned angle of the grille with the body is large, the grille is temporarily held in the air cleaner body by fitting the turning arm to the fitting parts. Further a photocatalytic holder containing a photocatalytic substance is provided in the air cleaner. A light emitting diode (LED) is provided for radiating light on the photocatalytic substance. Illumination of the LED is controlled in accordance with an actual voltage and the rated voltage of a power source.

### CROSS-REFERENCE TO RELATED APPLICATIONS

The present application is based on Japanese Patent Applications No. 2000-291989, No. 2000-291991, No. 2000-291994, No. 2000-292009 and No.2000-292015, which are incorporated herein by reference.

### BACKGROUND OF THE INVENTION

1. Field of the Invention

The present invention relates to an air cleaner that sucks air with a fan and houses a filter for removing a foul substance included in air and cleaning the air in a filter housing. More specifically, the invention relates to an air cleaner attached to the ceiling in the interior of a vehicle for example. Further the present invention relates to a photocatalytic filter having a photocatalytic substance and a light emitting diode illuminating device as well as an air cleaner provided with the photocatalytic filter and the light emitting diode illuminating device.

2. Description of the Related Art

The related art will be described using an air cleaner attached to the ceiling in the interior of a vehicle as an example below. Recently, in the interior of a vehicle, an air cleaner for cleaning air in the vehicle is provided. This air cleaner is provided with the body having a suction port and a blowout port and having an air passage between both, a fan arranged in the body and forming air flow from the suction port toward the blowout port in the air passage and an absorption part arranged in the air passage.

In an air cleaner, since a grille can be damaged when the grille directly falls while the grille is detached from the body, such the falling of the grille is not desirable. For technique for inhibiting the direct falling of a grille, technique for preventing a suction grille from falling by hanging the suction grille from the body with a stay when the suction grille is detached from the body in a ceiling flush type air conditioner is disclosed in JP-A-11-148673 (publication date: Jun. 2, 1999).

Also, in JP-A-10-103708 (publication date: Apr. 21, 1998), technique for providing a shank to a fitting piece formed so that the fitting piece can be reciprocated by a slit on both sides of a suction grille and fitting this shank into an opening hole of the body in a ceiling flush type air conditioner is disclosed.

According to prior art related to the former, as the air conditioner has structure that the suction grille is hung from the body with the stay to prevent the suction grille from falling, the stay is required to be detached to completely detach the suction grille from the body and it is troublesome.

According to prior art related to the latter publication, to detach the suction grille from the body, the shank is required to be detached from the opening hole by bending the fitting piece provided on both sides in the direction of the width of the grille. In this case, both fitting pieces provided on both sides in the direction of the width of the grille are required to be respectively bent and it is troublesome.

A photocatalytic substance is activated when light is radiated, has a function of dissolving and removing an organic substance as time elapses and is represented by titanium oxide (TiO₂). Such a photocatalytic substance is being used to dissolve and remove pollutant such as odorant. Such a photocatalytic substance is provided in the form of a particle. In industry, multiple particles of a photocatalytic substance are used and a trial of using a photocatalytic filter in which the particles are stuffed into a frame is made as disclosed in JP-A-9-38487. According to the invention, as air to be cleaned can pass between particles, contact between a particle of a photocatalytic substance and air is secured and air can be satisfactorily cleaned.

However, in a photocatalytic filter in which multiple particles of a photocatalytic substance are stuffed into a frame, depending upon used environment, a problem that multiple particles of a photocatalytic substance rattle, thereby, the particles collide, the particle collides with the wall of the frame, noise is made and makes a driver and others feel a sense of incompatibility occurs. Particularly, in case the photocatalytic filter is used in environment on which vibration frequently acts as in a vehicle, noise caused by the above-mentioned collision is remarkable.

FIG. 28 depicts a conventional embodiment of an air conditioning system disclosed in JP-A-7-315044 (publication date: Dec. 5, 1995). As shown in FIG. 28, the air conditioning system includes an air passage 111 and a filter housing 112, with filter 114, disposed in part of the air passage 111. Filter 114 is used to remove pollutants from an air flow which is formed in the air passage 111 by, for example, a fan (not shown).

In the technique of this patent application, the filter housing 112 itself is provided in the air passage 111 in which air flows. However, a filter insertion path for inserting the filter 114 into the filter housing 112 is provided as another path independent of the air passage 111 in which air flows. That is, as shown in FIG. 28, a filter port 210 is formed in a place different from the path of the air passage 111. The filter port 210 and the filter housing 112 are connected by a slit 117 formed on the wall 116a of a cylinder 116 of the filter housing 112 to communicate the filter port 210 and the filter housing 112 and space 118 provided under the slit 117. As described above, the space 118 forming the filter insertion path and the filter port 210 are provided on another path independent of the air passage 111 in which air flows.

In the technique of the patent application, the thin filter 114 is inserted into the filter port 210 in a crossing direction to a direction in which the air passage 111 is extended (a direction perpendicular to the page space of FIG. 28), that is, in a direction crossing the flow of air (a direction shown by an arrow Xl). Further, the filter 114 is inserted into the filter housing 112 in the direction in which the air passage 111 is extended (the direction perpendicular to the page space), that is, in the direction crossing the flow of air (a direction shown by an arrow X2) via the slit 117 utilizing the space 118, turning the filter.

According to prior art shown in FIG. 28, the filter insertion path for inserting the filter 114 into the filter housing 112 is a dedicated path and is a path independent of the air passage 111 in which air flows. Such the prior art large-sizes the whole. In this case, it is not desirable for a system attached to a place to be made compact such as the interior of a vehicle.

When the size H of the filter port 210 is reduced to make it compact, it becomes difficult to turn and insert or detach the filter 114. To simplify the insertion/detachment of the filter 114, the height and the size H of the filter port 210 have only to be increased, however, the whole is large-sized.

When this air cleaner is attached to the ceiling of the interior of a vehicle, it is general to bolt it and the air cleaner is securely fixed by bolting with another hand, supporting the air cleaner with one hand.

However, as it is general to bolt the conventional type air cleaner when it is attached to the ceiling as described above and the body of the air cleaner is securely fixed by bolting with one hand, supporting the body of the air cleaner with another hand, attachment work is not easy for the operator.

On the other hand, a light emitting diode illuminating device is provided with a diode that emits light, is small-sized and light in weight and is recently widely being used. Generally, a resistor for limiting current is connected to the light emitting diode for protection.

In the air cleaner provided with the photocatalytic filter of the photocatalytic substance, which cleans air flow and removes odorant in the air by activating the photocatalytic substance of the photocatalytic filter, a trial of activating the photocatalytic substance by light radiated from the light emitting diode is recently made.

It is therefore demanded to secure the luminous energy of the light emitting diode. To secure the luminous energy of the light emitting diode, a method of using in a state in which the voltage is high and increasing average current supplied to the light emitting diode can be adopted. However, excessive current is apt to be supplied to the light emitting diode, the heat of the light emitting diode becomes excessive and protective performance for the light emitting diode is deteriorated.

Further, the actual voltage of a power source supplying power to the light emitting diode may vary. Particularly, in the case of a power source for a vehicle, a frequency in which the actual voltage of the power source varies is high depending upon a situation of the vehicle. In case the actual voltage of the power source varies so that it is higher, excessive current is supplied to the light emitting diode, the heat of the light emitting diode becomes excessive and protective performance for the light emitting diode is deteriorated.

### SUMMARY OF THE INVENTION

The invention is made in view of such a situation and has an object of providing an air cleaner wherein a grille is temporarily held and the natural fall of the grille can be inhibited when the grille is detached from the body of the air cleaner and the grille can be detached from the body of the air cleaner with one hand of an operator by simple operation.

The invention also has an object of providing a photocatalytic filter that can contribute to the reduction of noise caused by the collision of particles of a photocatalytic substance and an air cleaner.

Further, the invention has an object of providing an air cleaner wherein a filter can be inserted or detached into/from a filter housing, turning the filter, in addition, an air passage and a filter insertion path can be made common and the whole size is made compact.

Still further, the invention has an object of providing an air cleaner the attachment work of which is simplified and particularly, which is suitable for attaching to an attached part on the side of the ceiling.

Furthermore, the invention has an object of providing a light emitting diode illuminating device advantageous to enhance protective performance for the light emitting diode, securing the luminous energy of the light emitting diode, a photocatalytic unit and an air cleaner.

An air cleaner according to the invention is based upon an air cleaner provided with the body of the air cleaner including a suction port, a blowout port, an air passage connecting both, a fan for forming air flow flowing from the suction port to the blowout port via the air passage and a cleaning part that cleans air flow flowing in the air passage and a grille covering at least one of the suction port and the blowout port of the body of the air cleaner so that ventilation is possible and attached to the body of the air cleaner so that the grille can be turned and can be detached and is characterized in that the grille has a turning arm provided to one end of the grille and functioning as the center of turning of the grille and a grille fitting part provided to the other end of the grille and fitted to the body of the air cleaner so that the grille fitting part can be detached, the body of the air cleaner has a first fitting part and a second fitting part mutually close and opposite via space, the first fitting part and the second fitting part of the body of the air cleaner enable the turning arm of the grille to be inserted or detached into/from the space between the first fitting part and the second fitting part when the turned angle of the grille with the body of the air cleaner is small and when the turned angle of the grille with the body of the air cleaner is large, the grille is temporarily held in the body of the air cleaner by fitting the turning arm of the grille to the second fitting part and fitting a part of the back of the surface fitted to the second fitting part of the grille turning arm to the first fitting part, keeping the grille in a turned position.

According to the invention, when the fan is driven, air sucked from the suction port flows in the air passage, is cleaned by the cleaning part and is blown out from the blowout port. The body of the air cleaner has the first fitting part and the second fitting part mutually close and opposite via the space. The first fitting part and the second fitting part of the body of the air cleaner enable the turning arm of the grille to be inserted or detached into/from the space between the first fitting part and the second fitting part when the turned angle of the grille with the body of the air cleaner is small. As described above, when the grille is pulled out in a state in which the insertion and detachment are enabled, the turning arm of the grille can be simply detached from the space between the first fitting part and the second fitting part.

The first fitting part and the second fitting part of the body of the air cleaner temporarily hold the grille in the body of the air cleaner, keeping the grille in a turned position by fitting the turning arm of the grille to the second fitting part and fitting the part of the back of the surface fitted to the second fitting part of the turning arm of the grille to the first fitting part when the turned angle of the grille with the body of the air cleaner is large. As described above, if the grille is temporarily held, the direct fall of the grille when the grille is detached from the body of the air cleaner is inhibited. Therefore, under a condition that the above-mentioned functions can be fulfilled, the first fitting part has only to be touched and fitted to one surface of the turning arm of the grille and the second fitting part has only to be touched and fitted to the other surface of the turning arm of the grille. The first fitting part and the second fitting part may be also in the shape of a pawl or a shank and the shape is not particularly limited.

The body of the air cleaner may be composed of the body including the suction port, the air passage, the fan and the cleaning part and a decorative plate attached to the body. In this case, the decorative plate can be provided with the first fitting part and the second fitting part. The decorative plate may be a suction grille.

The cleaning part may be an absorbing part that can absorb at least one of dust and odorant. For such an absorbing part, at least one of a filter for collecting dust or a photocatalyst can be adopted. For a filter, a non-woven fabric filter made of resin such as PP and PET or a glass fiber filter may be used. It is desirable that as the larger the surface area of a filter is, the more desirable it is to collect dust, the filter has a corrugated shape in which peaks and troughs are alternately repeated. It is also desirable that if necessary, an electrification process is executed. For example, the filter can be formed by laminating a layer mainly made of at least one of activated carbon, zeolite and silica gel and a layer to which an electrification process is applied. The filter may be also integrated with a photocatalyst and may be also not integrated with a photocatalyst. In case the filter is integrated with a photocatalyst, a filter acquired by changing photocatalyst powder to slurry and impregnating a filter with it by application, dipping and others can be adopted or photocatalyst powder may be also applied to the material of the filter. A photocatalyst is effective to remove odorant such as tobacco and a well-known substance such as TiO₂, WO₃, CdS, SrTiO₃ and MOS₂ can be used. Considering the simplicity of handling and the degree of activation, it is desirable that TiO₂ is used. For the crystal structure of TiO₂, both rutile structure and an anatase type can be used, however, it is generally desirable that the anatase type the catalytic activity of which is large is used. The shape of a photocatalyst is not particularly limited and a photocatalyst may be in various shapes and size such as powder and a particle. In this case, a photocatalytic substance itself may be also power or a particle or a photocatalyst may be also applied to a carrier in a state of powder or a particle. For the carrier, a particle of silica gel and activated carbon can be given, however, it is not limited to these.

The photocatalyst holder holding a photocatalyst may be also mounted on the grille. In this case, when the grille is detached from the body of the air cleaner, the photocatalyst holder can be detached from the body of the air cleaner. The photocatalyst holder can be detached from the grille. This case is advantageous to replace a photocatalyst held in the photocatalyst holder.

In an embodiment that a photocatalyst is provided on the downstream side of the filter, exciting means such as a light emitting diode can be also arranged on the downstream side of the filter. In this case, as dust and others are removed from air flow flowing in the air passage by the filter, the adhesion of dust and others to the exciting means such as a light emitting diode is inhibited, this embodiment is advantageous to protect the exciting means such as a light emitting diode and the capability of the exciting means such as a light emitting diode can be maintained for a long term.

In case the filter and the photocatalyst are separately provided in the air cleaner as separate cleaning parts, the photocatalyst may be also arranged on the downstream side of the filter or on the upstream side, however, as a large particle can be captured by the filter, it is desirable that the photocatalyst is arranged on the downstream side of the filter, considering that the adhesion of a large particle to the photocatalyst is inhibited and the performance of the photocatalyst is enhanced. For exciting means for exciting the photocatalyst, a light emitting diode, a black light and a cold-cathode tube can be adopted. For the light emitting diode, the one that emits light of a short wavelength (360 to 400 nm) can be adopted, however, the wavelength is not limited to the above-mentioned wavelengths. A light emitting diode made of gallium nitride (GaN) can be used, however, the light emitting diode is not limited to this. As a light emitting diode is a small-sized device and does not generally require much space for installation, it is advantageous to downsizing. As the light emitting diode emits at low voltage and small current, it is advantageous to power saving.

A photocatalytic filter according to the invention is provided with a frame having a housing and a ventilation window communicating with the housing, multiple particles of a photocatalytic substance stuffed in the housing formed by the frame and a mesh member attached to the ventilation window of the frame and having multiple mesh openings which a particulate in smaller size than a particle can pass and is characterized in that the mesh member is pressed on the stuffed particles to inhibit the rattle of particles.

An air cleaner according to the invention is based upon an air cleaner provided with a suction port, a blowout port, an air passage connecting both, a fan for forming air flow flowing from the suction port to the blowout port via the air passage and a cleaning part arranged in the air passage for cleaning air flow flowing in the air passage and is characterized in that the cleaning part is provided with the photocatalytic filter.

According to the photocatalytic filter according to the invention, as the mesh member attached to the ventilation window of the frame forming the photocatalytic filter is flexible in view of the property, it can be deflected in a direction in which particles are stuffed densely in the housing. As described above, particles can be stuffed densely in the housing utilizing the flexibility of the mesh member, the mesh member directly or indirectly presses the stuffed particles and the rattle of the particles is inhibited. Therefore, noise due to the rattle of the particles is also inhibited.

Ventilation is secured by the mesh openings of the mesh member and contact between the particle of a photocatalytic substance in the photocatalytic filter and air to be cleaned is secured. Further, as the mesh opening of the mesh member is smaller than the particle, the particle stuffed in the housing is prevented from falling from the mesh member. The mesh member has multiple mesh openings, may be also formed by braiding cotton and by punching multiple holes according to circumstances. In case cotton is braided, it may be also braided lengthwise and crosswise or cotton may be also extended only lengthwise or only crosswise.

The types of cotton for forming the mesh member are not particularly limited, may be also made of metal such as carbon steel, stainless steel, an aluminum alloy and titanium and may be also made of resin, however, to secure the strength of wire, reducing the diameter of the wire to enhance ventilation, metal is desirable. The shape of the mesh opening of the mesh member is not particularly limited and may be also square or circular.

According to a desirable embodiment of the photocatalytic filter, an elastic member that comes in contact with the particle can be stuffed in the frame together with the particles. In this case, as the elastic member is stuffed in the housing together with the particles, the rattle of the particles stuffed in the housing is more inhibited. The elastic member can be in the shape of a sheet, a mesh or a particle for example. It is desirable that the elastic member enables ventilation in case it has large surface area. To secure ventilation, the elastic member can be porous and for example, can be a mesh or porous layer having multiple minute openings enabling ventilation. In view of the fall of the particle, it is desirable that the size of the opening is smaller than that of the particle. The shape of the opening is not particularly limited and may be also square or circular. The elastic member can be formed using soft polymeric material such as resin and rubber.

The shape of the particle of a photocatalytic substance is not particularly limited and may be spherical, elliptical, cylindrical or prismatic. For the particle, a photocatalytic substance itself may also form a particle or the particle may be also formed by applying a photocatalytic substance to a catalyst support via binder. For the catalyst support, a silica gel particle and activated carbon can be given, however, the catalyst support is not limited to these. The size of the particle of a photocatalytic substance is different depending upon a purpose, for the upper limit value, 30 mm, 20 mm, 10 mm and 5 mm can be given and for the lower limit value, 0.1 mm, 0.5 mm, 1 mm and 3 mm can be given, however, the size is not limited to these.

According to the air cleaner according to the invention, when the fan is driven, air sucked from the suction port flows toward the blowout port via the air passage and is blown out from the blowout port. As pollutant included in air flow flowing in the air passage adheres to the particles of a photocatalytic substance of the photocatalytic filter forming the cleaning part, the air is cleaned. In the air cleaner according to the invention, as the photocatalytic filter having the above-mentioned structure is also mounted, the particles of a photocatalytic substance can be stuffed densely in the housing by utilizing the flexibility of the mesh member attached to the frame of the photocatalytic filter, the rattle of the particles is inhibited and noise caused by the rattle of the particles is also inhibited. As the mesh member has the mesh opening smaller in size than that of the particle, the particles stuffed in the housing are prevented from falling from the mesh member even if the particles are stuffed densely in the housing.

According to the desirable embodiment of the air cleaner according to the invention, a grille mounted on at least one of the suction port and the blowout port is provided and the above-mentioned photocatalytic filter and at least one of the grilles can have an attachment blocking part that blocks the attachment of the photocatalytic filter to the grille when the inside and outside of the photocatalytic filter are reversed. Hereby, the reverse attachment of the photocatalytic filter to the grille can be blocked and the capability of the photocatalytic filter can be normally utilized.

An air cleaner according to the invention is based upon an air cleaner provided with an air passage extended in the air cleaner, a filter housing provided in a part of the air passage, a fan for forming air flow flowing in the air passage and a filter housed in the filter housing for sticking pollutant included in the air flow and is characterized in that a filter port forming a part of the air passage and a concave portion communicating with the filter housing and provided on the opposite side to the filter port via a center line in the direction of the air flow in the filter housing for promoting the turning of the filter are provided and the filter is inserted or detached into/from the filter housing by moving the filter in a direction in which the air passage is extended from the filter port, turning the filter using the concave portion.

According to the invention, the filter port forming a part of the air passage and the concave portion provided on the opposite side to the filter port via the center line along air flow in the filter housing for promoting the turning of the filter are provided. The filter is inserted or detached via the filter port by moving the filter in the direction in which the air passage is extended (that is, from the upstream side of air flow to the downstream side or the downstream side of air to the upstream side), turning the filter using the concave portion.

As described above, as the filter is inserted or detached in the direction in which the air passage is extended, a filter insertion path can be formed by the air passage itself of the air cleaner. Therefore, differently from the prior art, a dedicated filter insertion path is not required to be provided separately from the air passage. As described above, in the air cleaner according to the invention wherein the filter insertion path is formed by the air passage itself, the blowout port on the downstream side of the air passage can also function as the filter port. Depending upon a case, the suction port on the upstream side of the air passage may also function as the filter port under a condition that the filter to be inserted or detached and the fan do not interfere with each other.

The filter port can be formed with it tilted for the center line in the direction of air flow in the filter housing. In this case, as the filter is inserted or detached diagonally for the filter housing, the insertion/detachment of the filter is easy.

The concave portion can have a guide part for assisting the turning of the filter on at least one side of the upstream side and the downstream side of the filter housing. Further in detail, at the upstream end of the filter housing in the concave portion for promoting turning, a first guide part having a tilt gradually toward the filter housing upstream can be formed. At the downstream end of the filter housing, a second guide part having a tilt toward the filter housing downstream can be formed. The concave portion can be provided with at least one of the first guide part and the second guide part.

For the filter, non-woven fabric made of resin such as PP and PET or a glass fiber filter can be used. As it is desirable that the surface area of the filter is larger to collect dust and others, it is desirable that the filter is in a corrugated shape in which peaks and troughs are alternately repeated. It is also desirable that if necessary, an electrification process is applied. The filter can be formed by laminating a layer mainly made of at least one of activated carbon, zeolite and silica gel and a layer to which an electrification process is applied.

The filter may be also provided with a photocatalyst or may be also provided with no photocatalyst. In case the filter is provided with a photocatalyst, a f ilter acquired by changing powder photocatalyst to slurry and impregnating it by application or dipping can be adopted or powder photocatalyst may be also applied to the material of a filter.

An air cleaner according to the invention is based upon an air cleaner provided with the body having a suction port and a blowout port and having an air passage between both, a fan arranged in the body for forming air flow from the suction port to the blowout port in the air passage and an absorbing part arranged in the air passage and is characterized in that the body is provided with an inserting pawl member protruded at the end of the body and inserted into an inserting hole of an attachment portion, a pushing pawl member pushed into a pushing hole of the attachment portion across a direction in which the inserting pawl member is inserted and a regularly fixing part regularly fixed to the attachment portion, the body is temporarily held in the attachment portions by inserting the inserting pawl member of the body into the inserting hole of the attachment portion and pushing the pushing pawl member of the body into the pushing hole of the attachment portion across the direction in which the inserting pawl member is inserted and after temporary holding, the regularly fixing part of the body is regularly fixed in the attachment portion.

According to the invention, the body of the air cleaner is temporarily held in the attachment portions by first inserting the inserting pawl member of the body into the inserting hole of the attachment portion, moving the pushing pawl member of the body in a direction that crosses the direction in which the inserting pawl member is inserted and pushing it into the pushing hole of the attachment portion. After temporary holding, the regularly fixing part of the body of the air cleaner is regularly fixed to the attachment portion. As described above, as the body of the air cleaner is regularly fixed after the body of the air cleaner is temporarily held in the attachment portions, the performance of attachment work is improved. Particularly, as the natural fall of the body of the air cleaner in a temporarily held state is inhibited in case the attachment portion is located on the ceiling, workability in regular fixing is improved. Therefore, even if the attachment portion is located on the ceiling of the interior of a vehicle, attachment work is facilitated. It is desirable that the inserting pawl member is provided at the end of the body and the pushing pawl member is provided at the back end of the body.

The inserting pawl member has a function of being inserted into the inserting hole of the attachment portion and a function for supporting the inserting pawl member of the body in an inserted state and the shape is free. The pushing pawl member has a function of being pushed into the pushing hole of the attachment portion and a function for supporting the pushing pawl member of the body in a pushed state and the shape is free.

The attachment portion may be located on the ceiling of the interior of a vehicle. In this case, for the attachment portion, a reinforcement part which is a reinforcing member on the side of the ceiling can be adopted, however, this embodiment is not limited to this. In the case of a vehicle, the reinforcement part is generally metallic.

The body may be provided with a grille that functions as at least one of a suction port and a blowout port and a nipping part for nipping a part of a decorative wall together with the grille. In this case, it may be set that the outer end of the grille bites a part of the decorative wall together with nipping. Hereby, the peeling and others of the terminal of the decorative wall are inhibited. Further, as the outer end of the grille bites the decorative wall, difference in a level in a boundary between the decorative wall and the grille is easily reduced and the appearance of the boundary is improved. It is desirable that the outer end of the grille is curved so that it is protruded in a direction of biting.

A light emitting diode illuminating device according to the invention is based upon a light emitting diode illuminating device provided with a light emitting diode connected to a power source and fed from the power source, a switching circuit for turning on or off the light emitting diode and a control circuit that controls the switching circuit and is characterized in that the control circuit executes continuous control that the switching circuit is controlled so that the light emitting diode is continuously turned on when the actual voltage of the power source does not exceed the rated voltage area of the power source and executes pulse control that time when the light emitting diode is turned on is reduced when the actual voltage of the power source exceeds the rated voltage area of the power source.

A photocatalytic converter according to the invention is based upon a photocatalytic converter provided with a photocatalytic substance and an activating part for activating the photocatalytic substance and is characterized in that the activating part is constituted by the foregoing light emitting diode.

An air cleaner according to the invention is based upon an air cleaner provided with a suction port and a blowout port, an air passage connecting both, a fan for forming air flow from the suction port to the blowout port via the air passage and a photocatalytic filter arranged in the air passage and provided with a photocatalytic substance for cleaning air flow in the air passage and an activating part for activating the photocatalytic substance of the photocatalytic filter and is characterized in that the activating part is constituted by the foregoing light emitting diode.

According to the light emitting diode illuminating device according to the invention, when the actual voltage of a power source does not exceed the rated voltage area of the power source, continuous control that the switching circuit is controlled so that the light emitting diode is continuously turned on is executed. When the actual voltage of the power source exceeds the rated voltage area of the power source, pulse control that time in which the light emitting diode is turned on is reduced is executed. Therefore, as mean current per unit time supplied to the light emitting diode is inhibited even if the actual voltage of the power source exceeds the rated voltage area of the power source, the excessive heat of the light emitting diode is inhibited, the durability of the light emitting diode is enhanced and the life is extended.

Further, according to the light emitting diode illuminating device according to the invention, as control is made so that the light emitting diode is continuously turned on when the actual voltage of the power source does not exceed the rated voltage area of the power source, the luminous energy of the light emitting diode is secured. As pulse control that time in which the light emitting diode is turned on is reduced is executed when the actual voltage of the power source exceeds the rated voltage area of the power source, it can be set that when the actual voltage of the power source is in the rated voltage area of the power source, mean current supplied to the light emitting diode is a maximum current area. Therefore, in a general used state high in the frequency of use, the light emitting diode can emit in a full or substantially full state and the luminous energy of the light emitting diode can be increased. The rated voltage area in the invention means an area in the vicinity of a rated voltage value. A rated voltage area generally means a range from +1% to −10% based upon a rated voltage area.

According to the light emitting diode illuminating device according to the invention, as described above, when the actual voltage of the power source does not exceed the rated voltage area of the power source, continuous control that the light emitting diode is continuously turned on is executed and when the actual voltage of the power source exceeds the rated voltage area of the power source, pulse control that time in which the light emitting diode is turned on is reduced is executed, however, when the actual voltage is in the rated voltage area of the power source, the above-mentioned continuous control may be also executed and the above-mentioned pulse control may be also executed.

For example, when a value of the rated voltage of the power source is 13.5 V (a threshold), continuous control that the light emitting diode is continuously turned on is executed in case the actual voltage of the power source is lower than the rated voltage of the power source or in case it does not exceed the rated voltage and pulse control that time in which the light emitting diode is turned on is reduced is executed in case the actual voltage of the power source exceeds the rated voltage of the power source.

According to the preferred embodiment of the light emitting diode illuminating device according to the invention, the control circuit controls so that when the x-axis shows the actual voltage of the power source and the y-axis shows mean current per unit time supplied to the light emitting diode, mean current increases until the actual voltage of the power source increases up to the rated voltage area of the power source, as the actual voltage of the power source exceeds the rated voltage area of the power source, mean current decreases and when the actual voltage of the power source is in the rated voltage area of the power source, mean current supplied to the light emitting diode is in a maximum current area. Hereby, when the actual voltage of the power source is in the rated voltage area of the power source, the capability of the emission of the light emitting diode can be made a full or substantially full state. Further, even if the actual voltage of the power source varies higher than the rated voltage area of the power source, the excessive heat of the light emitting diode can be inhibited and the light emitting diode can be more securely protected.

According to the preferred embodiment of the light emitting diode illuminating device of the invention, the control circuit is provided with the triangular wave generator that generates a triangular pulse having mean voltage corresponding to the actual voltage of the power source and the comparator that compares the voltage of a triangular pulse output from the triangular wave generator and reference voltage, the comparator outputs a signal for turning on the light emitting diode to the switching circuit when the voltage of the triangular pulse is lower than the reverence voltage and outputs a signal for turning off the light emitting diode to the switching circuit when the voltage of the triangular pulse is higher than the reference voltage. Hereby, even if the actual voltage of the power source varies higher than the rated voltage area of the power source, the excessive heat of the light emitting diode can be inhibited and the light emitting diode can be more securely protected.

A light emitting diode that emits light including ultraviolet rays having a wavelength of 360 to 400 nm can be adopted. A light emitting diode made of gallium nitride (GaN) can be used, however, the invention is not limited to this. The number of light emitting diodes may be also one or plural.

A photocatalytic converter according to the invention is provided with a photocatalytic substance and an activating part for activating the photocatalytic substance. The activating part is constituted by the above-mentioned light emitting diode. The photocatalytic substance is activated by radiating light from the light emitting diode on the photocatalytic substance and photocatalytic efficiency is enhanced. According to the photocatalytic converter according to the invention, the photocatalytic substance can be loaded on another member such as cloth, ceramics and metal. To load, binder can be used.

An air cleaner according to the invention is provided with a suction port and a blowout port, an air passage connecting both, a fan for forming air flow from the suction port to the blowout port via the air passage, a photocatalytic filter arranged in the air passage and having a photocatalytic substance for cleaning air flow flowing in the air passage and an activating part for activating the photocatalytic substance of the photocatalytic filter. The activating part is constituted by the above-mentioned light emitting diode. According to the air cleaner of the present invention, when the fan is driven, air sucked from the suction port flows to the blowout port via the air passage and is blown out from the blowout port. As pollutant included in air flow flowing in the air passage adheres to the photocatalytic substance of the photocatalytic filter, air is cleaned. The pollutant stuck on the photocatalytic substance is gradually dissolved and removed. The activating part for activating the photocatalytic substance of the photocatalytic filter includes the light emitting diode, the photocatalytic substance is activated by radiating light on the photocatalytic substance of the photocatalytic filter and air cleaning efficiency is enhanced.

According to the photocatalytic converter of the invention and the air cleaner of the invention, as described above, the activating part includes the light emitting diode. Therefore, when the actual voltage of a power source does not exceed the rated voltage area of the power source, continuous control that the switching circuit is controlled so that the light emitting diode is continuously turned on is executed and when the actual voltage of the power source exceeds the rated voltage area of the power source, pulse control that time in which the light emitting diode is turned on is reduced is executed. Therefore, even if the actual voltage of the power source exceeds the rated voltage area of the power source, the heat of the light emitting diode is inhibited, the light emitting diode can be more securely protected, the durability of the light emitting diode is enhanced, the life is extended and the photocatalytic substance can be activated for a long term.

Further, according to the photocatalytic converter of the invention and the air cleaner of the invention, as control is made so that the light emitting diode is continuously turned on when the actual voltage of a power source does not exceed the rated voltage area of the power source, the luminous energy of the light emitting diode is secured and the above-mentioned photocatalytic converter and air cleaner are advantageous to activate the photocatalytic substance. Further, as mean current supplied to the light emitting diode can be set when the actual voltage of the power source is in a rated voltage area of the power source so that it is in a maximum current area, the light emitting diode can emit in a full or substantially full state in a general used state high in the frequency of use, that is, when the actual voltage of the power source is in a rated voltage area of the power source and the photocatalytic substance can be satisfactorily activated.

### BRIEF DESCRIPTION OF THE DRAWINGS

FIG. 1 is a sectional view showing an air cleaner equivalent to one embodiment of the invention;

FIG. 2 is an enlarged sectional view showing the vicinity of a filter and a photocatalyst holder of the air cleaner equivalent to one embodiment of the invention;

FIG. 3 is a plan showing the air cleaner equivalent to one embodiment of the invention;

FIG. 4 is an enlarged sectional view showing a blowout grille turned downward in one embodiment of the invention;

FIG. 5 is an enlarged sectional view schematically showing a process that a filter is inserted or detached into/from a filter housing in the body of the air cleaner equivalent to one embodiment of the invention;

FIG. 6 is a partial enlarged sectional view showing a state in which a turning arm of the blowout grille is fitted to a first fitting part and a second fitting part in case the turned angle of the blowout grille is large;

FIG. 7 is a partial enlarged sectional view showing a state in which the turning arm of the blowout grille can be inserted or detached into/from space between the first fitting part and the second fitting part in case the turned angle of the blowout grille is small;

FIG. 8 is a partial enlarged sectional view showing the photocatalyst holder mounted on the blowout grille and the vicinity of exciting means;

FIG. 9 is a partial enlarged view showing a mesh member;

FIG. 10 is a partial enlarged view showing a mesh member;

FIG. 11 is a side view showing a state immediately before the photocatalyst holder is mounted on the blowout grille;

FIG. 12 is a side view showing a state after the photocatalyst holder is mounted on the blowout grille;

FIG. 13 shows a section viewed along a line W17-W17 in FIG. 12 and is a sectional view showing the vicinity of a holder fitting part in the state after the photocatalyst holder is mounted on the blowout grille;

FIG. 14 is a plan showing a photocatalytic filter;

FIG. 15 is a side view showing the photocatalytic filter;

FIG. 16 is a perspective view schematically showing the mounting structure of the air cleaner equivalent to one embodiment of the invention;

FIG. 17 is an enlarged sectional view showing a state in which an inserting pawl member is inserted into an inserting hole of a first reinforcement part on the side of the ceiling in one embodiment of the invention;

FIG. 18 is an enlarged sectional view showing a state in which a pushing pawl member is inserted into a pushing hole of a second reinforcement part on the side of the ceiling in one embodiment of the invention;

FIG. 19 is a plan showing the fitting of the pushing pawl member to a fitting piece of the body in one embodiment of the invention;

FIG. 20 is a circuit diagram showing a light emitting diode illuminating device;

FIG. 21 shows a waveform of a triangular pulse generated by a triangular wave generator;

FIG. 22 shows a situation in which a switching device is operated and a situation in which the light emitting diode (LED) is operated when a triangular pulse is smaller than reference voltage together with a waveform;

FIG. 23 shows a situation in which the switching device is operated and a situation in which the light emitting diode is operated when the maximum voltage of a triangular pulse is close to the reference voltage together with a waveform;

FIG. 24 shows a situation in which the switching device is operated and a situation in which the light emitting diode is operated when the maximum voltage of a triangular pulse exceeds the reference voltage together with a waveform; 15 FIG. 25 shows a situation in which the switching device is operated and a situation in which the light emitting diode is operated when the maximum voltage of a triangular pulse considerably exceeds the reference voltage together with a waveform;

FIG. 26 shows a situation in which the switching device is operated and a situation in which the light emitting diode is operated when the maximum voltage of a triangular pulse further exceeds the reference voltage together with a waveform;

FIG. 27 is a graph schematically showing relation between the actual voltage of a power source and mean current supplied to the light emitting diode; and

FIG. 28 is a sectional view schematically showing a process in which a filter is inserted or detached into/from a filter housing according to prior art.

### DETAILED DESCRIPTION OF THE PREFERRED EMBODIMENTS

Referring to the drawings, embodiments acquired by embodying the invention will be described below.

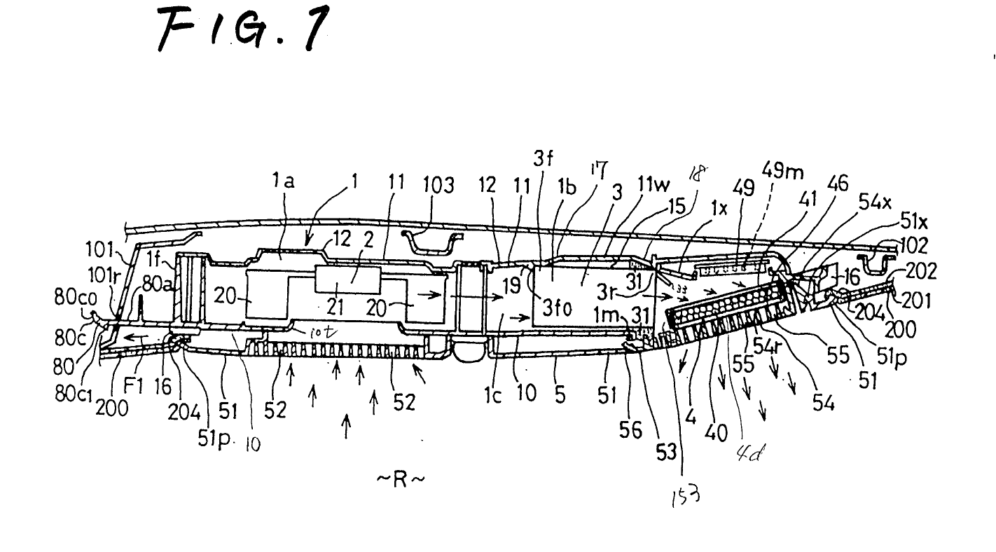

As shown in FIG. 1, an air cleaner equivalent to this embodiment is attached to the ceiling of the interior of a vehicle such as an automobile, and is provided with the thin, that is, flat body 1, a fan 2 arranged in a fan chamber 1a of the body 1, a filter 3 for collecting dust which is arranged in a filter housing 1b in the body 1 and functions as a first absorbing part, a photocatalyst holder 4 housing a photocatalyst 40 which is arranged in the body 1 and functions as a second absorbing part and a suction grille 51 made of resin as a decorative plate mounted on the lower surface of the body 1 so as to be attachable and detachable. As shown in FIG. 1, the body 1 has a box-type body 12 formed by integrating a front plate 10 having a suction port 10t on the side of the interior R of a car body and a back plate 11 on the side of the cat body and a decorative laminated grille 5 mounted on the lower surface of the body 12 so that the decorative laminated grille is opposite to the interior R and can be detached.

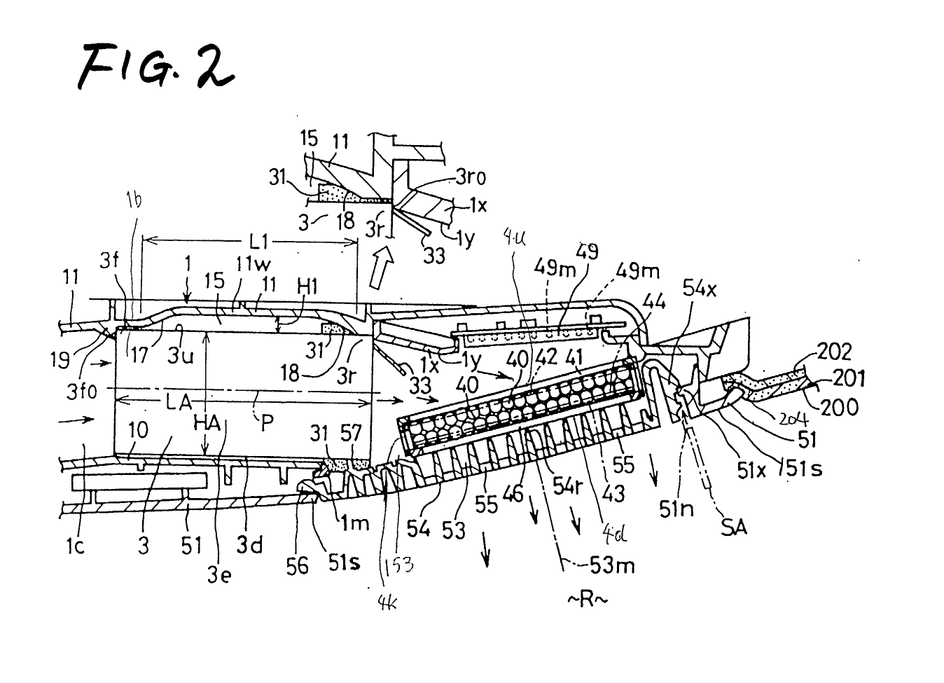

As shown in FIG. 1, the decorative laminated grille 5 is provided with the suction grille 51 covering a half or larger area of the lower surface of the body 12 of the body 1 and forming multiple grille suction ports 52 and a blowout grille 54 mounted in an opening 153 for mounting provided on the downstream side of the suction grille 51 so as to be attachable and detachable. In the suction grille 51, a blowout port 53 also functioning as a filter port is formed. As shown in FIG. 2, this blowout port 53 is downward based upon the center line P in a direction of air flow of the filter housing 1b. The reason is that as the air cleaner equivalent to this embodiment is attached to the ceiling of the interior R, cleaned air is blown out from the blowout port 53 toward the lower interior R. As shown in FIG. 2, the normal 53m of the blowout port 53 inclines in a direction apart from the filter housing 1b in the lower part of the blowout port. The blowout port 53 can also function as a filter port for inserting or extracting the filter 3 described later in addition to a function of blowing out cleaned air flow toward the interior R.

The above-mentioned suction grille 51 is mounted on the lower surface of the body 1 so that the suction grille is opposite to the interior R and can be detached. The blowout grille 54 is made of resin, is opposite to the interior R and has multiple grille blowout ports 55. The size of the blowout grille 54 is smaller than that of the suction grille 51. The grille blowout port 55 communicates with the filter housing 1b and the interior R. When the blowout grille 54 is attached in the opening 153 for mounting of the suction grille 51, a fitting pawl 56 at one end of the blowout grille 54 is fitted to a fitting part 51s on the downstream side of the suction grille 51 and a grille fitting pawl 54x at the other end of the blowout grille 54 is fitted to a grille fitting pawl 51x of a downstream part 151s of the suction grille 51 so that they can be detached as understandable from FIG. 2.

As shown in FIG. 1, an air passage 1c is formed between the grille blowout port 55 and the grille suction port 52 in the body 1. In the air passage 1c, the fan 2 opposite to the grille suction port 52, the filter 3 for mainly collecting dust and others and the photocatalyst holder 4 for mainly removing the smell of tobacco and others are arranged successively from the upstream side to the downstream side.

The fan 2 shown in FIG. 1 is a centrifugal fan such as a multi-vane radial fan, is provided with plural moving vanes 20 and a driving part 21 for driving the moving vanes 20 and sucks air in the interior R of the car body via the grille suction port 52.

A concave portion 15 is formed by recessing the wall part 11w of the back plate 11 in a direction in which the volume of the filter housing 1b is increased as shown in FIG. 1 in the filter housing 1b out of the back plate 11 forming the body 1. The concave portion 15 is arranged on the upside of the filter housing 1b and as understandable from the following description, can function as a turning promoting space for turning the filter 3. As understandable from FIG. 2, the wall part lw forming the concave portion 15 out of the back plate 11 is extended along the center line P of the filter housing 1b along air flow. As understandable from FIG. 2, the concave portion 15 is provided on the opposite side to the blowout port 53 which is also the filter port via the center line P of the filter housing 1b. As shown in FIG. 2, the depth H1 of the concave portion 15 is set so that the depth is considerably smaller than the height HA of the filter 3. The length L1 of the concave portion 15 is slightly shorter than the length LA of the filter 3. At the upstream end of the filter housing 1b out of the concave portion 15, a first guide part 17 having a tilt gradually downward (toward the filter housing 1b) upstream is formed. At the downstream end of the filter housing 1b, a second guide part 18 having a tilt downward (toward the filter housing 1b) downstream is formed.

The filter 3 is housed in the filter housing 1b so that the filter can be detached, is mainly a dust collection filter for collecting dust, is formed by weaving flexible thin filter material that passes air in the shape of pleats so that peaks 3m and troughs 3n are alternately formed as shown in FIG. 3, the whole appearance is substantially in the shape of a rectangular box (length: LA, height: HA, width: DA, see FIGS. 2 and 3) and as shown in FIG. 2, has the upper surface 3u, the lower surface 3d and the side 3e. The filter material forming the filter 3 has laminated structure acquired by laminating a layer mainly made of activated carbon and a layer to which an electrification process is applied to enhance the performance of dust collection. As shown in FIG. 2, the end face 3fo of the upstream end 3f of the filter 3 is fitted to a filter upstream fitting part 19 of the back plate 11 of the body 1, hereby, they seal to prevent air flowing in the air passage 1c from entering the concave portion 15 and positions the filter 3 in a direction in which the filter is inserted. The filter upstream fitting part 19 is extended in the direction (a direction shown by an arrow E) of the width of the body 1 as shown in FIG. 3 and seals the whole width or the substantial whole width of the end face 3fo of the upstream end 3f of the filter 3.

As shown in FIG. 2, the end face 3ro of the downstream end 3r of the filter 3 is slightly fitted to a filter downstream fitting part 1x held by the back plate 11 of the body 1, hereby, also positions the filter 3 in the direction in which the filter is inserted and prevents the rattle of the filter 3. As shown in FIG. 2, a tilt guide surface 1y which is the lower surface of the filter downstream fitting part 1x is downward tilted toward the blowout grille 54 downstream and has a function of promoting operation for tilting the inserted/detached filter 3.

Further, on the lower surface 3d, the upper surface 3u and the side 3e of the downstream end 3r of the filter 3, cushion material 31 for enhancing sealing performance and more inhibiting the rattle of the filter 3 is held. That is, as shown in FIG. 2, as the cushion material 31 on the upper surface 3u of the filter 3 is in contact with the downstream end of the concave portion 15, the sealing performance of the upper surface part 3u of the filter 3 is satisfactorily maintained and the rattle of the filter 3 is more prevented. As shown in FIG. 2, as a pressing convex portion 57 of the blowout grille 54 is pressed into the cushion material 31 on the lower surface 3d of the filter 3 when the blowout grille 54 is closed, the sealing performance of the lower surface part 3d of the filter 3 is satisfactorily maintained and the rattle of the filter 3 is inhibited more.

As described above, as the filter 3 is housed in the filter housing 1b in a sealed state, air flowing in the air passage 1c is prevented from passing the concave portion 15 and securely passes the filter 3. A tab 33 which can function as a filter tension member is integrated on the upside of the downstream end 3r of the filter 3. When the tab 33 is pulled with fingers, the filter 3 in the filter housing 1b can be pulled out on the side of the blowout port 53.

The configuration of the vicinity of the photocatalytic holder 4 which is a main part will be described below. In this embodiment, as shown in FIG. 2, the photocatalytic holder 4 is held on the back side 54r of the blowout grille 54 so that the photocatalytic holder can be detached. Therefore, when the blowout grille 54 is detached from the blowout port 53 of the suction grille 51 attached to the body 1, the photocatalytic holder 4 also similarly automatically detached from the body 1. Further, as the photocatalyst holder 4 can be also detached from the blowout grille 54, the configuration is advantageous to replace the photocatalyst 40 in the photocatalyst holder 4. The photocatalytic holder 4 is a deodorizing filter mainly for deodorization, the lower surface 4d is opposite to the blowout grille 54 on the downstream side of the filter 3 and the upper surface 4u is opposite to the exciting means 49. As shown in FIG. 8, the photocatalytic holder 4 is formed by a first frame 41 and a second frame 46 respectively integrated so that they form a housing 4t and they can be separated in the shape of a thin box. In the photocatalytic holder 4, multiple photocatalytic particles 40 are stuffed in the housing 4t densely to prevent rattle. Clearance between adjacent photocatalytic particles 40 functions as an air passage. As shown in FIG. 8, in the flat photocatalytic holder 4, multiple photocatalytic particles 40 are arranged in the direction of the surface of the photocatalytic holder 4 to enhance the performance of the catalyst, however, the number of the photocatalytic particles 40 arranged in the direction of the thickness of the photocatalytic holder 4 is reduced to secure ventilation. In the above-mentioned photocatalytic holder 4, as shown in FIG. 8, a ventilation window 41x for passing air is formed in the substantially whole region of the upper surface 4u of the first frame 41. A ventilation window 46x for passing air is formed in the whole region of the lower surface 4d of the second frame 46.

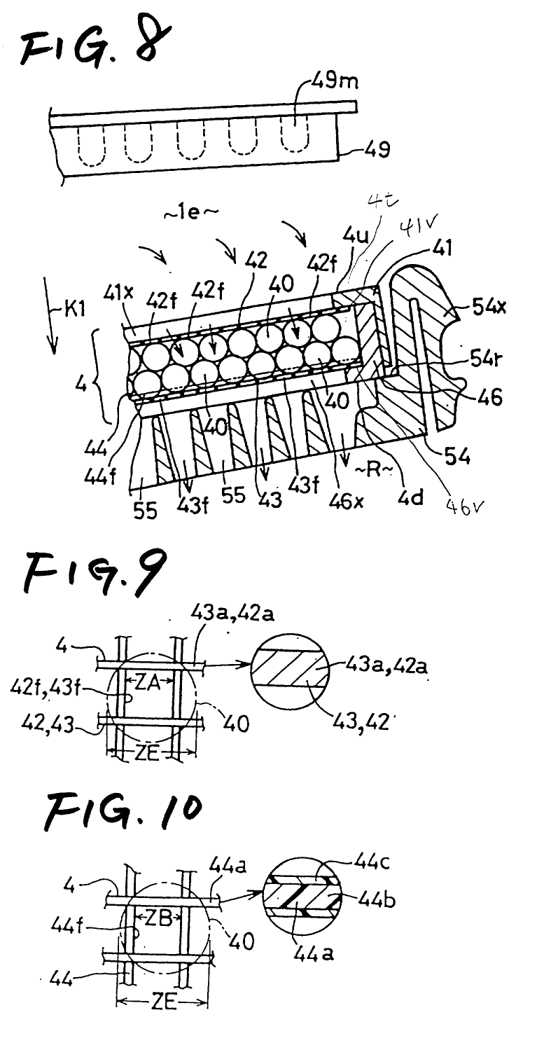

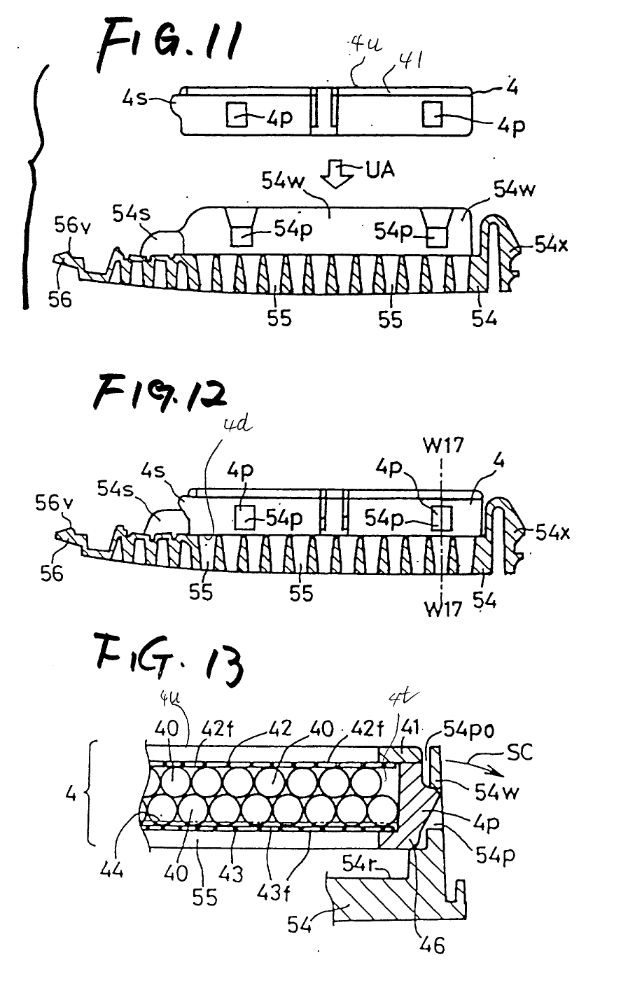

The photocatalytic particle 40 is in the shape of a minute ball (diameter: 2 to 6 mm for example) and is mainly made of titanium oxide (TiO₂) which is aphotocatalytic substance. The diameter of each photocatalytic particle 40 is substantially the same. As shown in FIG. 8, on the side opposite to the exciting means 49 out of the photocatalytic holder 4, ametallic mesh member 42 having multiple mesh openings 42f is opposed to the ventilation window 41x of the first frame 41 and is touched to the inner surface 41v of the first frame 41 to prevent detachment. As also shown in FIG. 8, on the side with the back to the exciting means 49, an elastic member 44 made of polymeric material having multiple mesh openings 44f coated with rubber and resin and a metallic mesh member 43 having multiple mesh openings 43f are opposed to the ventilation window 46x of the second frame 46 under the photocatalytic particles 40. As shown in FIG. 8, the elastic member 44 and the mesh member 43 are touched to the inner surface 46v of the second frame 46 to prevent detachment. In order farther from the exciting means 49, that is, in order in a direction (a direction in which air flow passes) shown by an arrow K1 in FIG. 8, the metallic mesh member 42, the photocatalytic particles 40, the elastic member 44 made of polymeric material and the metallic mesh member 43 are arranged. As shown in FIG. 9, the metallic mesh members 42 and 43 are formed by braiding metallic thin wire 42a and 43a in a mesh. As shown in FIG. 10, as the elastic member 44 made of polymeric material and having a thin diameter is formed by braiding wire 44a acquired by coating a core 44b made of nylon and others with a soft layer 44c made of urethane and others in a mesh, it is excellent in protective performance for the photocatalytic particles 40. As understandable from FIGS. 9 and 10, the diameter ZA of each mesh opening 42f, 43f of the metallic mesh members 42 and 43 and the diameter ZB of the mesh opening 44f of the elastic member 44 made of polymeric material are substantially the same and are set so that both are smaller than the outside diameter ZE of the photocatalytic particle 40. Therefore, the photocatalytic particle 40 stuffed in the housing 4t is prevented from falling from each mesh opening 42f, 43f of the mesh members 42 and 43 of the photocatalytic holder 4 and the mesh opening 44f of the elastic member 44. In such a photocatalytic holder 4, photocatalytic particles in the shape of a minute ball can be stuffed in the housing 4t of the photocatalytic holder 4 densely, preventing the photocatalytic particles in the shape of a minute ball from falling, the rattle of the photocatalytic particles in the shape of a minute ball is inhibited and the passing of air is also secured. As the elastic member 44 is coated with polymeric material such as rubber and resin, it is advantageous to inhibit the rattle of photocatalyst 40 in the shape of a particulate.

FIG. 14 is a plan showing the photocatalytic holder 4. FIG. 15 is a side view showing the photocatalytic holder 4. As shown in FIG. 15, the photocatalytic holder 4 is in the shape of a thin, that is, flat rectangular box thinned in a direction of ventilation to reduce the resistance of ventilation and multiple side ventilation windows 4k are formed on the side of the photocatalytic holder 4. The performance of ventilation is more enhanced by the side ventilation window 4k and the cleaning performance of the photocatalytic holder 4 can be more enhanced. The first frame 41 and the second frame 46 are connected by fitting a fitting pawl 41w formed on the first frame 41 of the photocatalytic holder 4 to the second frame 46 so that the first and second frames can be separated. As shown in FIG. 14, a crossed arm 41r is laid across the ventilation window 41x formed on the upper surface 4u of the first frame 41. The arm 41r can function as an excessive deflection inhibiting part for securing the strength of the first frame 41 and further, inhibiting the excessive deflection of the mesh member 42, securing the opening area of the ventilation window 41x. An arm that can prevent the detachment of the mesh member 43 is also similarly laid across the ventilation window 46x of the second frame 46 though it is not shown.

The exciting means 49 shown in FIG. 8 is provided to excite the photocatalytic particles 40 in the photocatalytic holder 4 and is held over the photocatalytic holder 4 so that the exciting means is opposite to the upper surface 4u of the photocatalytic holder 4. In the exciting means 49, multiple light emitting diodes 49m are arranged. The exiting means 49 is thin, that is, flat along the flat photocatalytic holder 4 as a whole. The light emitting diodes 49m are made of a gallium nitride semiconductor (peak of emission wavelength: approximately 370 to 400 nm), radiates light of a short wavelength on the photocatalytic particles 40 and is exposed in the air passage 1c. When light from the light emitting diodes 49m is radiated, the photocatalytic particle 40 is excited and activated and the action of dissolving a substance such as odorant which adheres to the photocatalytic particle 40 is accelerated. As shown in FIGS. 1 and 2, as the exciting means 49 is arranged on the downstream side of the filter 3 for removing dust, the adhesion of dust included in air flow to the light emitting diodes 49m is inhibited, it is advantageous to secure the luminous energy of the light emitting diodes 49m for a long term and in addition, it is also advantageous to secure the cleaning performance of the photocatalytic particles 40 activated by light radiated from the light emitting diodes 49m.

Structure in which the photocatalytic holder 4 is attached to the blowout grille 54 will be described below. As shown in FIG. 11, plural filter fitting parts 4p are protruded on the side of the photocatalytic holder 4. When the photocatalytic holder 4 is moved to the blowout grille 54 in a direction shown by an arrow UA and is pressed on the blowout grille, the filter fitting part 4p of the photocatalytic holder 4 is fitted to a filter fitted part 54p of the blowout grille 54 and the photocatalytic holder 4 is attached to the blowout grille 54 so that it can be detached. As the projection 4s of the photocatalytic holder 4 becomes in contact with the projection 54s of the blowout grille 54 when the inside and the outside of the photocatalytic holder 4 are reversed, the photocatalytic holder 4 cannot be attached to the blowout grille 54. Therefore, the photocatalytic holder 4 is attached to the blowout grille 54 so that the elastic member 4 coated with the polymeric material layer is always located on the side of the lower surface of the photocatalytic holder 4. Therefore, the projections 4s and 5s can function as an attachment blocking part for preventing the inside and the outside of the photocatalytic holder 4 from being reversed. Therefore, the interception of light radiated from the light emitting diodes 49m by the elastic member 44 is inhibited. Therefore, this embodiment is advantageous to activate the photocatalytic particles 40 by light radiated from the light emitting diodes 49m and is advantageous to secure the capability of the photocatalytic holder 4. As the elastic member 44 is always arranged on the side far from the light emitting diodes 49m, the deterioration of the elastic member 44 by radiation can be also inhibited.

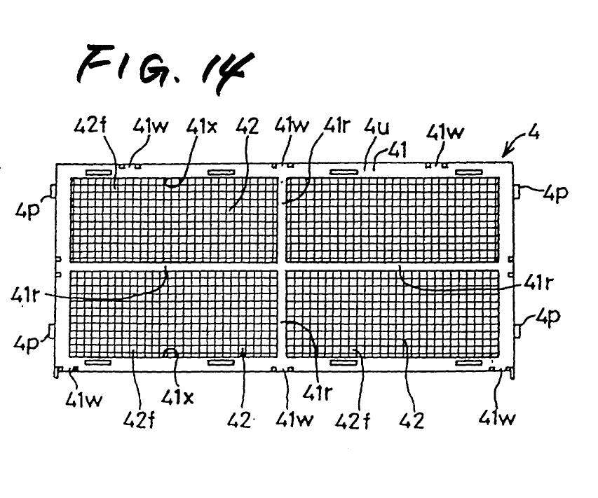

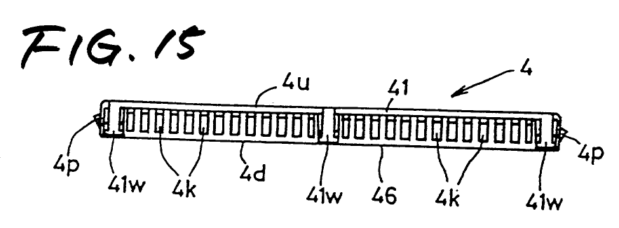

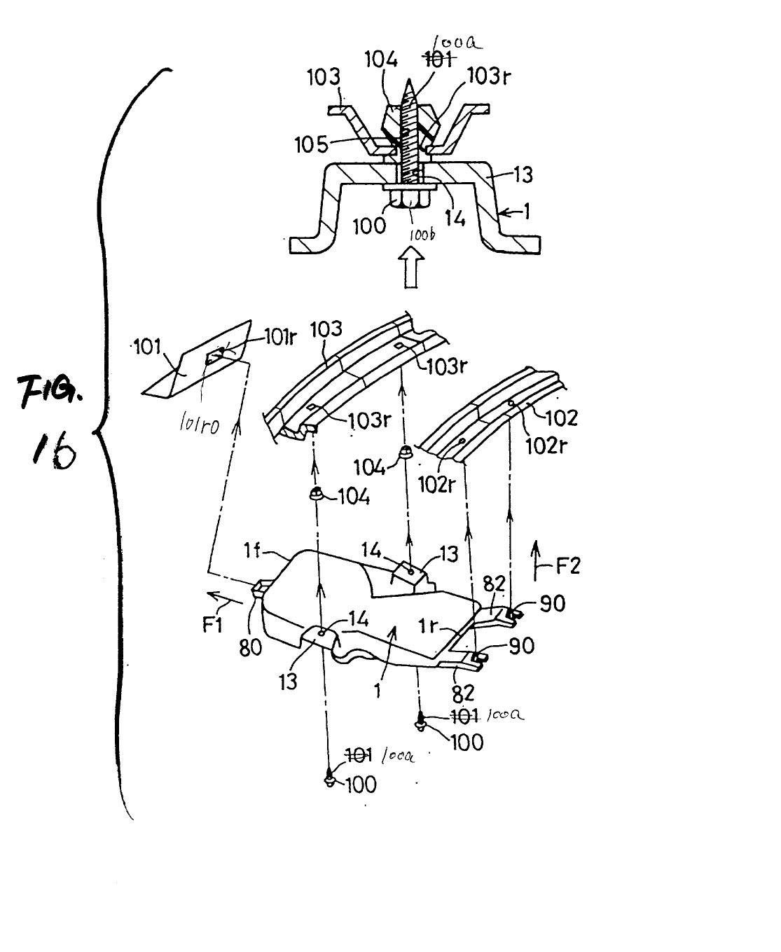

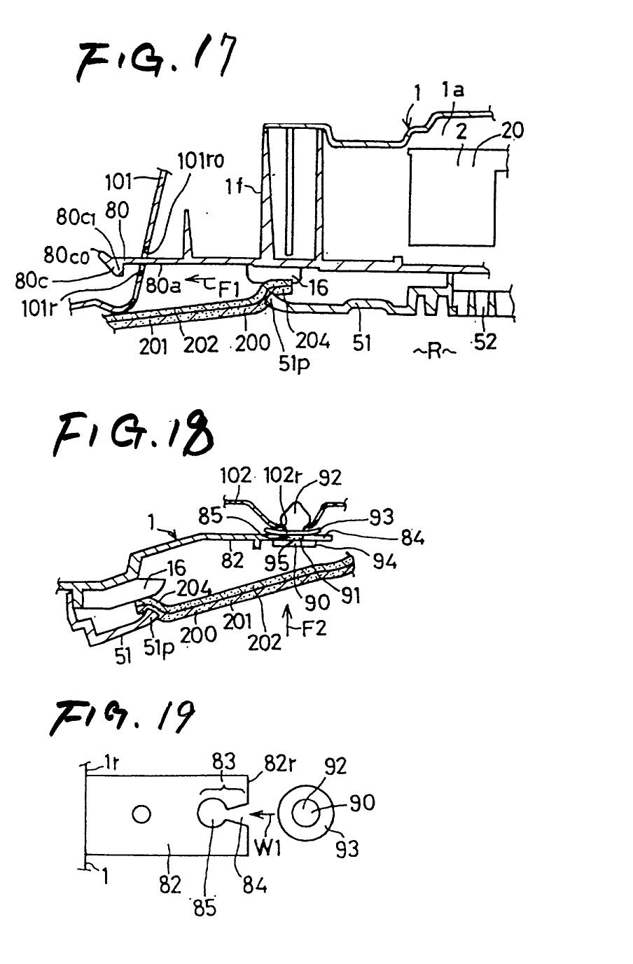

As shown in FIG. 13, as the wall 54w of the blowout grille 54 having the filter fitted part 54p is elastically deformed in the same direction when an inserting member such as a tool and a coin is inserted into clearance 54po between the filter fitting part 4p and the filter fitted part 54p and is pressed outside, that is, in a direction shown by an arrow SC, fitting between the filter fitting part 4p of the photocatalytic holder 4 and the filter fitted part 54p of the blowout grille 54 is released and the photocatalytic holder 4 is detached from the blowout grille 54. The frequency of the detachment of the photocatalytic holder 4 is smaller than that of the filter 3 for collecting dust.

Next, a case in which the air cleaner is used will be described below. The fan 2 is rotated in a state shown in FIG. 1, the light emitting diodes 49m of the exciting means 49 are emitted and the photocatalytic particles 40 are activated. Air in the interior R of a vehicle is sucked from the suction port 10t via the grille suction port 52 of the suction grille 51 by the rotation of the fan 2, the air flows in the air passage 1c and is cleaned via the filter 3 and the photocatalytic particles 40 in the order. The cleaned air is blown out into the interior R from the grille blowout port 55 of the blowout grille 54 via the blowout port 53. The interior R is gradually cleaned by the continuation of such operation. Generally, a relatively large particle such as dust in air is collected by the filter 3 and a extremely minute particulate such as odorant included in air is absorbed in the photocatalytic particles 40. An organic substance stuck on the photocatalytic particle 40 is gradually dissolved and removed. Therefore, the filter 3 and the photocatalytic holder 4 can both function as a cleaning part for cleaning air flow.

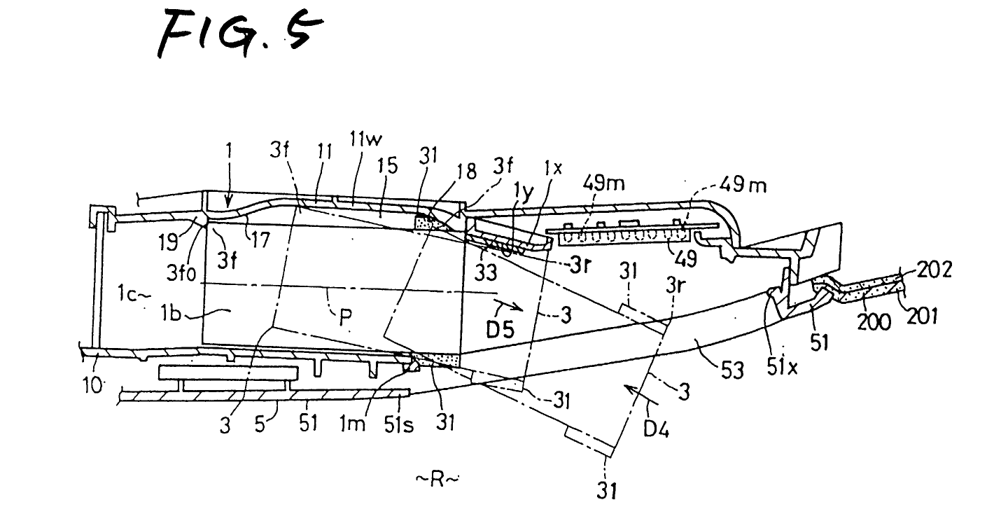

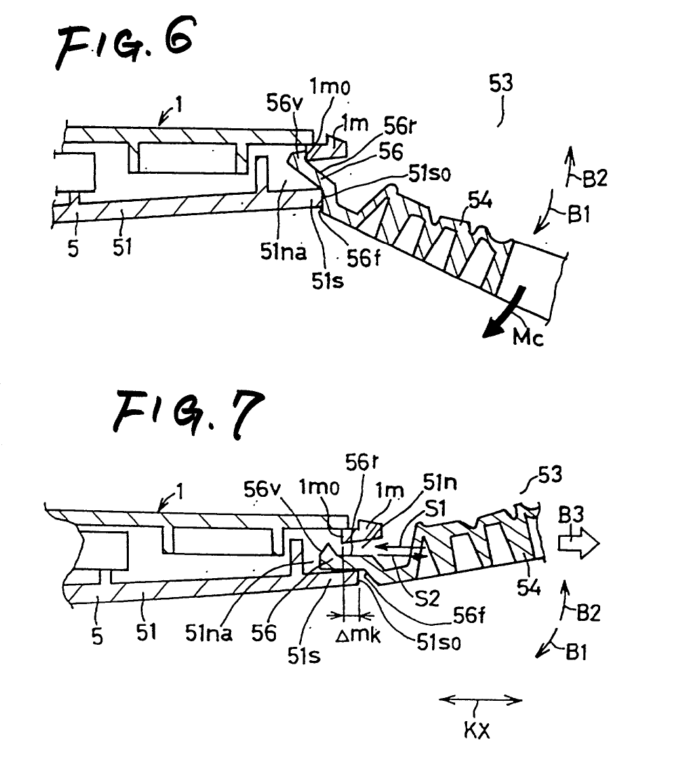

While the air cleaner is used for a long term, a case that the blowout grille 54 is detached from the blowout port 53 of the body 1 to replace the filter 3 or the photocatalyst 40 with new one may occur. In this case, when the blowout grille 54 falls from the body 1 carelessly, the blowout grille 54 is damaged. Then, in this embodiment, the following characteristic configuration is adopted. That is, as shown in FIGS. 6 and 7, a turning arm 56 having a protruded fitting head 56v at the end is protruded at one end of the blowout grille 54. On the downstream side of the suction grille 51 forming a part of the body 1, a first fitting part 1m and a second fitting part 51s mutually close and opposite via space 51na are provided. As shown in FIGS. 6 and 7, for the first fitting part 1m and the second fitting part 51s, the second fitting part 51s is arranged on the side (the side of the surface of the suction grille 51) of a direction in which the blowout port is opened in a direction in which the blowout grille 54 is turned and the blowout port 53 is opened or closed (a direction shown by an arrow B1 or B2) and the first fitting part lm is arranged on the side (the back side of the suction grille 51) of a direction in which the blowout port is closed. As shown in FIG. 7, the fitting end face 1mo of the first fitting part 1m and the fitting end face 51so of the second fitting part 51s are directed in reverse directions in a direction shown by an arrow KX which is a direction in which the blowout grille 54 is extended and are set off by Δ mk. That is, the fitting end face 1mo of the first fitting part lm and the fitting end face 51so of the second fitting part 51s are apart in the direction shown by the arrow KX which is the direction in which the blowout grille 54 is extended. Therefore, the fitting end face 51so of the second fitting part 51s is arranged on the side closer to the blowout grille 54 than the fitting end face 1mo of the first fitting part 1m in the direction shown by the arrow KX. The fitting end face 1mo of the first fitting part 1m is arranged on the side farther from the blowout grille 54 than the fitting end face 51so of the second fitting part 51s in the direction shown by the arrow KX.

As shown in FIG. 7, when an angle of the turning of the blowout grille 54 with the body 1 is small, the turning arm 56 of the blowout grille 54 can be inserted or detached into/from space 51na between the first fitting part lm and the second fitting part 51s in a direction shown by an arrow S1 or S2. As shown in FIG. 6, when an angle of the turning of the blowout grille 54 with the body 1 is large, the turning arm 56 of the blowout grille 54 is fitted to the fitting end face 51so of the second fitting part 51s and a part of the back 56r of the surface 56f fitted to the second fitting part 51s out of the turning arm 56 of the blowout grille 54 is fitted to the first fitting part 1m. In this state, as understandable from FIG. 6, the turning arm 56 of the blowout grille 54 is fitted in structure supported by two points of the first fitting part 1m and the second fitting part 51s in a turned state. Therefore, the blowout grille 54 in the turned state is temporarily held by the body 1 as it is in the turned state without naturally falling. That is, as shown in FIG. 6, clockwise moment Mc acts on the blowout grille 54 by the self-weight of the blowout grille 54 and the blowout grille 54 tries to turn clockwise with the turning arm 56 in the center of turning. However, as the first fitting part 1m that functions as a stopper is fitted to the turning arm 56 of the blowout grille 54, the turning of the blowout grille 54 is stopped in the position. As a result, the blowout grille 54 is temporarily held in the turned state without naturally falling. Therefore, even if an operator separates his/her fingers from the blowout grille 54 in the turned state when the blowout grille 54 is detached, the natural fall of the blowout grille 54 is prevented and the damage of the photocatalyst holder 4 and the photocatalyst 40 caused by the natural fall of the blowout grille 54 can be prevented beforehand.

As it is conceivable that the turning arm 56 of the blowout grille 54 having weight to some extent is more strongly fitted to the first fitting part 1m, compared with a case of the too light blowout grille 54, the temporarily held posture of the blowout grille 54 is stable. At this point, in this embodiment, as the blowout grille 54 is integrated with the photocatalyst holder 4 housing the photocatalytic particles 40, it has weight to some extent and the temporarily held posture is stabilized.

In addition, in this embodiment, as shown in FIGS. 6 and 7, as a fitting head 56v is provided at the end of the turning arm 56 and the fitting head 56v is fitted to the first fitting part lm, the turning arm 56 and the first fitting part 1m can be more securely latched and the turned blowout grille 54 is temporarily held more securely.

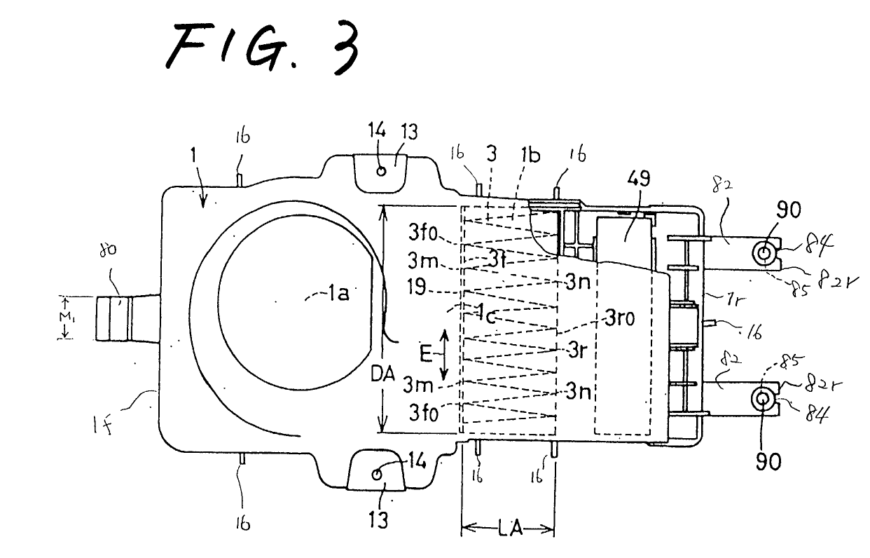

The description will be further continued below. In case the photocatalyst 40 and the filter 3 are replaced, the grille fitting pawl 54x as a grille fitting part is provided at the other end of the blowout grille 54 as shown in FIG. 2, an inserting member SA such as a tool, a coin and the end of a finger is inserted into clearance 51n between the grille fitting pawl 54x and the grille fitting pawl 51x of the suction grille 51 and is suitably turned. Hereby, at least one of the grille fitting pawls 54x and 51x is suitably elastically deformed and fitting between the grille fitting pawl 54x and the grille fitting pawl 51x is released. Then, as shown in FIG. 4, the blowout grille 54 with which the photocatalyst holder 4 is integrated lowers, turning in the direction shown by the arrow B1 by the self-weight with the vicinity of the turning arm 56 in the center of turning. In this state, as understandable from the description, the turning arm 56 of the blowout grille 54 is fitted to the first fitting part lm and the second fitting part 51s and as a result, the turned blowout grille 54 is temporarily held by the body 1 without naturally falling. Therefore, the first fitting part 1m can function as temporarily holding means for temporarily holding the blowout grille 54 in the body 1 in an open state. Afterward, as shown in FIG. 7, when the whole blowout grille 54 is slightly lifted in the direction shown by the arrow B2 (in a closed direction) substantially horizontally and an angle of the turning of the blowout grille 54 with the body 1 is reduced, fitting between the turning arm 56 of the blowout grille 54 and the first fitting part 1m and fitting between the turning arm and the second fitting part 51s are naturally released. As a result, the turning arm 56 can be inserted or detached into/from the space 51na between the first fitting part 1m and the second fitting part 51s. When the blowout grille 54 is moved in a direction shown by an arrow B3 (see FIGS. 7 and 4) in a state in which insertion and detachment are enable as described above, the blowout grille 54 can be simply detached from the suction grille 51 on the side of the body 1 and the blowout port 53 can be opened. The above-mentioned operation is enabled by only turning downward, slightly lifting the temporarily held blowout grille 54 and extracting the turning arm 56 from the space 51na and an operator can simply execute the operation with one hand.

After the blowout grille 54 is detached from the suction grille 51 as described above, the photocatalyst holder 4 is detached from the blowout grille 54. The detached photocatalyst holder is replaced with a new photocatalyst holder 4. The new photocatalyst holder 4 is attached to the blowout grille 54. Afterward, as described above, the turning arm 56 of the blowout grille 54 is inserted into the space 51na between the first fitting part 1m and the second fitting part 51s, the blowout grille 54 is turned upward in the direction shown by the arrow B2, the grille fitting pawl 54x of the blowout grille 54 and the grille fitting pawl 51x of the suction grille 51 are fitted as shown in FIG. 2 and the blowout grille 54 is attached to the suction grille 51 so that the blowout grille can be detached. The frequency of the detachment of the photocatalyst holder 4 having the photocatalyst 40 excited by the exciting means 49 is smaller, compared with that of the filter 3.

As shown in FIG. 1, the blowout grille 54 attached to the suction grille 51 on the side of the body and in a state in which the blowout port 53 is closed is tilted upward from the side of the turning arm 56 to the side of the grille fitting pawl 54x.

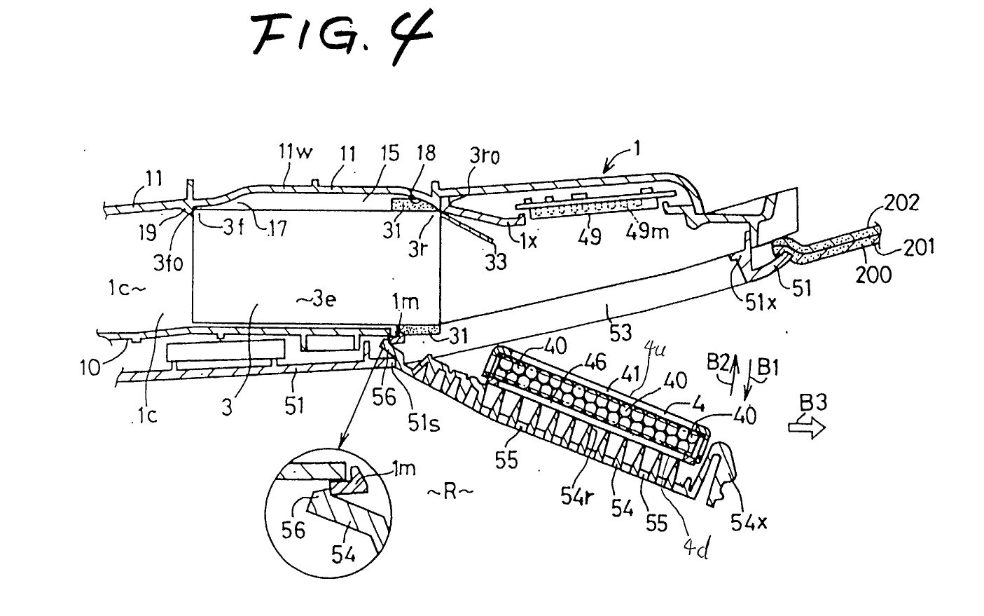

That is, as understandable from FIG. 5, the blowout grille 54 is detached from the blowout port 53 of the suction grille 51 of the body 1 and the blowout port 53 which is the filter port is opened. As shown in FIG. 5, the filter 3 is diagonally tilted so that the upstream end 3f is on the upside and the downstream end 3r is on the downside. The filter 3 in that state is inserted into the filter housing 1b first from the upstream end 3f in a direction shown by an arrow D4 via the blowout port 53 of the suction grille 51. At this time, the vicinity of the upstream end 3f of the filter 3 can be satisfactorily guided by the tilt guide action of the tilt guide face 1y of the filter downstream fitting part lx and the second guide part 18 of the concave portion 15.

Making the upside of the upstream end 3f of the filter 3 enter the concave portion 15, the filter 3 is turned, pushing the filter 3 until the filter is substantially horizontal along the flat filter housing 1b. As the concave portion 15 is formed opposite to the filter housing 1b, turning operation for turning the filter 3 diagonally tilted until it is substantially horizontal is easy. Particularly in this embodiment, when the upstream end 3f of the filter 3 hits the first guide part 17, the upstream end 3f of the filter 3 can be guided downward by the tilt guide action of the first guide part 17 and turning operation for turning the filter 3 sideways (substantially horizontally) is more facilitated.

After the filter 3 is attached in the filter housing 1b as described above, the blowout grille 54 is attached to the blowout port 53 of the suction grille 51 as described above. That is, as described above, the turning arm 56 of the blowout grille 54 is fitted to the first fitting pawl 1m of the suction grille 51, the blowout grille 54 is turned upward in the direction shown by the arrow B2, the grille fitting pawl 54x of the blowout grille 54 and the grille fitting pawl 51x of the suction grille 51 are fitted and the blowout grille 54 is attached to the suction grille 51.

Next, a case that the filter 3 is detached from the filter housing 1b of the body 1 will be described. In this case, basically, operation reverse to the case that the filter 3 is attached in the filter housing 1b has only to be executed and the filter 3 in the filter housing 1b is pulled and moved from the upstream side to the downstream side in the air passage 1c. That is, first, the blowout grille 54 is detached from the suction grille 51 of the body 1 and the blowout port 53 which is also the filter port is opened. As understandable from FIG. 5, the filter 3 is pulled out of the filter housing 1b, pulling the tab 33 connected to the upside of the downstream end 3r of the filter 3 in a direction shown by an arrow D5 toward the blowout port 53. At this time, the filter 3 is further pulled out, turning the filter so that the upside of the upstream end 3f of the filter 3 enters the concave portion 15 and the downstream end 3r of the filter 3 is diagonally downward. In this embodiment, as the concave portion 15 is formed in the upper part of the filter housing 1b, it is easy to perform turning operation, tilting the substantially horizontal filter 3 so that the upstream end 3f is on the upside and the downstream end 3r is on the downside also when the filter is pulled out.

Particularly in this embodiment, operation for diagonally turning the filter 3 is more facilitated by the tilt guide action of the second guide part 18 provided on the downstream side of the concave portion 15 and the tilt guide face 1y. After the filter is pulled out as described above, the filter 3 is cleaned or replaced. The cleaned or replaced filter 3 is attached in the filter housing 1b again as described above and the blowout grille 54 is attached to the blowout port 53 of the suction grille 51 fixed to the body 1.

Structure for mounting the air cleaner equivalent to this embodiment will be described below. As shown in FIGS. 1, 3 and 16, in the central area of the end 1f of the body 1 of the air cleaner, one inserting pawl member 80 is provided so that it is protruded along the surface of the body 1. The inserting pawl member 80 is wide and has a relatively large width dimension M1 (see FIG. 3). The inserting pawl member 80 is provided with an extended part 80a protruded transversely from the end if of the body 1 and an inserting head 80c protruded from the end of the extended part 80a and having a pawl function as shown in FIG. 17. As shown in FIG. 17, the inserting head 80c has a protruded part 80c0 protruded upward in a direction in which the inserting head is inserted and a protruded part 80c1 protruded in a reverse direction (downward) to the direction in which the protruded part 80c0 is protruded. That is, the inserting pawl member 80 has the protruded parts 80c0 and 80c1 on both sides in a direction which crosses the inserted direction. Hereby, the performance to prevent the detachment of the inserting pawl member 80 is enhanced. When the inserting pawl member 80 is moved together with the body 1 along the surface of the body 1, that is, in a direction shown by an arrow F1, it is inserted into a long sideway inserting hole lOlr of a first reinforcement part 101 on the side of the ceiling. The inserting hole 101r is pierced in a direction in which the inserting pawl member 80 is protruded from the body 1 (the inserting pawl member 80 is inserted).

As described above, in a state in which the inserting pawl member 80 is inserted into the inserting hole 101r, the position of the body 1 can be adjusted in a direction of the length of the body 1 (in the direction in which the inserting pawl member 80 is inserted). As there is a margin between the inner edge 101ro of the inserting hole 101r and the inserting pawl member 80, the position of the body 1 can be also adjusted in a direction of the height of the body 1. Further, the position of the body 1 can be also adjusted in a direction of the width of the body 1.

Further as shown in FIGS. 3 and 16, two mounting pieces 82 are protruded in parallel at the back end 1r of the body 1 of the air cleaner and a pushing pawl member 90 is attached to each mounting piece 82 so that the pushing pawl member can be detached. The total two pushing pawl members 90 are provided. As shown in FIG. 19, a holding opening 83 the back of which is open is formed in the mounting piece 82 protruded from the back end 1r of the body 1. This holding opening 83 is formed by a widened opening 84 widened toward the back end 82r of the mounting piece 82 and a circular opening 85 communicating with the widened opening 84. The pushing pawl member 90 is made of elastic material such as resin and as shown in FIG. 18, is provided with a shank 91, a pushing head 92 having a large diameter and provided at the end of the shank 91, a lip 93 extended in a direction of the diameter in the intermediate part of the shank 91, a detachment preventing part 94 extended in the direction of the diameter at the base of the shank 91 and a groove 95 formed between the lip 93 and the detachment preventing part 94. The pushing pawl member 90 is relatively displaced in a direction shown by an arrow W1 (see FIG. 19) and is inserted into the circular opening 85 via the widened opening 84. Hereby, the groove 95 of the pushing pawl member 90 is fitted to the mounting piece 82 and held. When the pushing pawl member 90 is moved together with the body 1 in a direction that crosses the direction in which the inserting pawl member 80 is inserted, that is, a direction shown by an arrow F2 (a direction of the height of the body 1), the pushing head 92 of the pushing pawl member 90 is forcedly pressed in a pushing hole 102r of a second reinforcement part on the side of the ceiling 102 and is fitted. The pushing hole 102r is pierced in a direction that crosses the direction in which the inserting pawl member 80 is inserted, that is, in the direction of the height of the body 1.

As described above, as there is a slight margin between the pushing pawl member 90 and the inner wall of the holding opening 83 of the mounting piece 82 in a state in which the pushing pawl member 90 is pushed into the pushing hole 102r, the position of the body 1 can be adjusted in a direction of the length of the body 1 and in a direction of the width. As shown in FIG. 3, the inserting pawl member 80 and the pushing pawl member 90 are provided at both ends in the direction of the length of the body 1 so that they turn their backs to each other.

Further, as shown in FIGS. 3 and 16, two regularly fixing parts 13 are provided at both ends in the direction of the width on the upper surface of the body 1 of the air cleaner. The regularly fixing part 13 is arranged between the inserting pawl member 80 and the pushing pawl member 90 in the direction of the length of the body 1. As understandable from FIG. 16, a regularly fixing hole 103r is formed on a third reinforcement part 103 on the side of the ceiling of the car body and is pierced in the direction that crosses the direction in which the inserting pawl member 80 is inserted, that is, in the direction of the height of the body 1. A clip 104 made of resin for fixing the body is pressed in the regularly fixing hole 103r and is held beforehand.

As shown in FIG. 16, in attachment, a male screw 100a having a tapping function and a fitting bolt 100 having a bolt head 100b are used. With the bolt head 100b downward, the fitting bolt 100 is inserted into an inserting hole 14 of the regularly fixing part 13 from the end and a female screw 105 is forcedly formed by tapping the clip 104 with the male screw 10a of the fitting bolt 100 by rotating the bolt head 100b of the fitting bolt 100 with a tool and others. Hereby, the male screw 10a of the fitting bolt 100 and the female screw 105 of the clip 104 screw. Therefore, the regularly fixing part 13 of the body 1 can be regularly fixed to the third reinforcement part 103 on the side of the ceiling of the car body.

As in such a regularly fixed state, the female screw 105 and the male screw 100a screw and are fastened, the required attachment strength of the body 1 of the air cleaner is secured. The above-mentioned first reinforcement part on the side of the ceiling 101, second reinforcement part on the side of the ceiling 102 and third reinforcement part on the side of the ceiling 103 are formed by a metallic member mounted on the car body for reinforcement and compose an attachment portion according to the invention.

The case that the air cleaner is attached on the side of the ceiling of the interior R of the car body will be further described below. In this case, suppose that the decorative laminated grille 5 formed by the suction grille 51 and the blowout grille 54 is detached from the body 1. As understandable from FIG. 16, the inserting pawl member 80 is inserted and fitted into the inserting hole 101r of the first reinforcement part on the side of the ceiling 101 on the side of the car body by moving the inserting pawl member 80 at the end if of the body 1 along the surface of the body 1, that is, in the direction shown by the arrow F1 (in the direction of the length of the body 1) together with the body 1. In this state, the back end 1r of the body 1 can be lifted or lowered in the direction of the height of the body 1.

The pushing head 92 of the pushing pawl member 90 is pressed and fitted in the pushing hole 102r of the second reinforcement part on the side of the ceiling 102 on the side of the car body by moving the back end 1r of the body 1 together with the pushing pawl member 90 in the direction that crosses the direction in which the inserting pawl member 80 is inserted, that is, in the direction shown by the arrow F2 (the direction of the height of the body 1). In this state, the body 1 of the air cleaner is temporarily held at three points by one inserting pawl member 80 and the two pushing pawl members 90.

As the inserting pawl member 80 in this embodiment is wide, the inserting pawl member 80 is more stably temporarily held at the rim of the inserting hole 101r of the first reinforcement part on the side of the ceiling 101, compared with a case that the width of the inserting pawl member is narrow and performance for temporarily holding the body 1 is enhanced. As in the above-mentioned temporarily held state, the position of the inserting pawl member 80 and the pushing pawl member 90 can be adjusted as described above, the regularly fixing hole 103r of the third reinforcement part 103 on the side of the ceiling of the car body and the inserting hole 14 of the regularly fixing part 13 of the body 1 can be easily aligned.

As described above, in a state in which the body 1 of the air cleaner is temporarily held and with the bolt head 100b of the fitting bolt 100 downward, the fitting bolt 100 is inserted into the inserting hole 14 of the regularly fixing part 13 and the bolt head 100b of the fitting bolt 100 is rotated by a tool and others. Hereby, the female screw 105 is formed by tapping the clip 104 with the male screw 10a of the fitting bolt 100, and the female screw 105 and the male screw 100a screw. Hereby, the regularly fixing part 13 of the body 1 of the air cleaner can be regularly fixed to the third reinforcement part on the side of the ceiling 103 which is an attachment portion of the side of the ceiling of the car body. As in such a state in which the body 1 of the air cleaner is regularly fixed, the inserting pawl member 80 is fitted into the inserting hole 101r of the first reinforcement part on the side of the ceiling 101 which is an attachment portion of the side of the car body and the pushing pawl member 90 is fitted into the pushing hole 102r of the second reinforcement part on the side of the ceiling 102, performance for preventing the detachment of the body 1 is more enhanced.

As described above, in this embodiment, as the body 1 of the air cleaner can be regularly fixed to the reinforcement part on the side of the ceiling 103 in a state in which the body 1 of the air cleaner is temporarily held in the reinforcement parts on the side of the ceiling 101 and 102 to prevent a natural fall, the body 1 of the air cleaner is not required to be supported by an operator or a robot and others in regularly fixing. Further, as it is also avoided that unnatural posture is enforced upon an operator or a robot and others, not only the operability of attachment can be enhanced but the safety of the operator can be enhanced.

A ceiling wall 200 shown in FIGS. 1 and 2 is also called roof lining, forms the upholstery wall of the interior R, is formed by a thin plate and is formed by a decorative surface layer 201 opposite to the interior R and a lined layer 202 stuck on the back of the surface layer 201.

As shown in FIG. 1, plural nipping parts 16 in the shape of a protruded rib are provided around the body 1. As described above, after the body 1 of the air cleaner is regularly fixed to the third reinforcement part on the side of the ceiling 103, a ceiling wall 200 is arranged around the body 1. Afterward, the decorative laminated grille 5 composed of the suction grille 51 and the blowout grille 54 is attached to the upper surface of the body 1. As a result, as shown in FIGS. 1, 2 and 17, the terminal 204 of the ceiling wall 200 existing around the body 1 is nipped by the outer end 51p of the suction grille 51 of the decorative laminated grille 5 and the nipping part 16 in a direction of the thickness of the terminal and the outer end 51p of the suction grille 51 is made to bite the terminal 204 of the ceiling wall 200 from the lower side of the terminal. Hereby, the terminal 204 of the ceiling wall 200 around the body 1 is more securely fixed and it is advantageous to prevent the terminal 204 of the ceiling wall 200 around the body 1 from being peeled. Further, as the outer end 51p of the suction grille 51 bites the terminal 204 of the ceiling wall 200, difference in a level in a boundary between the terminal 204 of the ceiling wall 200 which is decorative material of the interior R of the car body and the suction grille 51 is reduced, smoothness between the suction grille 51 and the terminal 204 of the ceiling wall 200 is enhanced and the appearance of the boundary can be improved. As shown in FIG. 3, as the multiple nipping parts 16 are provided around the body 1, the outer end 51p of the suction grille 51 bites in multiple places utilizing the nipping part 16 as described above and the appearance around the body 1 can be improved. To enhance the performance of the biting of the outer end 51p of the suction grille 51, the outer end 51p of the suction grille 51 is curved in a direction of the biting, that is, slightly upward.

In this embodiment, as the body 1, the exciting means 49 and the photocatalyst holder 4 are thin, this embodiment can contribute to the reduction of the thickness of the air cleaner, the air cleaner is also attached to the ceiling without a sense of incompatibility in the interior R of a vehicle and it is advantageous to realize the large interior R of the car body.

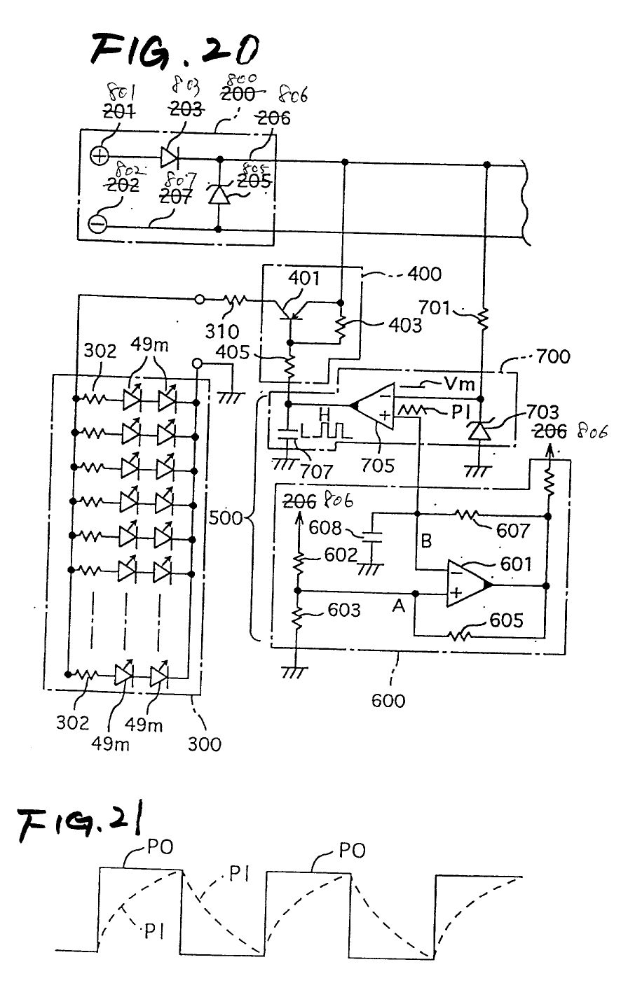

a light emitting diode illumination device equivalent to this embodiment will be described below. As shown in FIG. 20, the light emitting diode illuminating device is provided with a power circuit 800 connected to a power source, a group of light emitting diodes 300 including multiple light emitting diodes 49m fed from the power circuit 800, a switching circuit 400 for the on-off control of the group of light emitting diodes 300 and a control circuit 500 that controls the switching circuit 400. The power circuit 800 is provided with a power source terminal (a positive electrode) 801, a power source terminal (a negative electrode) 802 respectively connected to the power source, a diode 803, Zener diode 805 and electric supply lines 806 and 807.

The switching circuit 400 is provided with a PNP-type transistor 401 which is a switching device, a resistor 403 connected to the emitter and the base of the transistor 401 and a resistor 405 connected to the base to the transistor 401. The emitter of the transistor 401 is connected to a cathode of the diode 803 and is fed via the diode 803.

The control circuit 500 is provided with a triangular wave generator 600 that generates a triangular pulse P1 having mean voltage having magnitude corresponding to the actual voltage of the power source and a comparator 700 that compares the voltage of a triangular pulse and reference voltage Vm. The triangular wave generator 600 is provided with a comparator 601, resistors 602 and 603 for dividing voltage, a resistor 605 connected to a noninverting input terminal (+) of the comparator 601 and the output terminal of the comparator 601, a resistor 607 connected to an inverting input terminal (−) of the comparator 601 and the output terminal of the comparator 601 and a capacitor 608 connected to the output terminal and the inverting input terminal (−) of the comparator 601 via the resistor 607. The power supply of the power circuit 800 is divided by the resistors 602 and 603 and divided voltage is input to the noninverting input terminal (+) of the comparator 601. The output voltage of the comparator 601 is fed back to the inverting input terminal (−) of the comparator 601 via the resistor 607 and is fed back to the noninverting input terminal (+) of the comparator 601 via the resistor 605.

The comparator 700 is provided with Zener diode 703 connected to the power circuit via a resistor 701, a comparator 705 to the inverting input terminal (−) of which reference voltage Vm regulated by the resistor 701 and Zener diode 703 is input and a capacitor 707 connected to the output terminal of the comparator 705 and the base of the transistor 401. The output terminal of the comparator 705 is connected to the base of the transistor 401 via the resistor 405. A triangular pulse P1 output from the triangular wave generator 600 is input to the noninverting input terminal (+) of the comparator 705.

The group of light emitting diodes 300 is composed by connecting plural sets in parallel (for example, 10 to 60 sets) in one of which plural light emitting diodes 49m and a resistor 302 for limiting current are connected in series. The anode of the group of light emitting diodes 300 is connected to the collector of the transistor 401 via a resistor 310 for limiting current. The light emitting diodes 49m emit light including ultraviolet rays having a wavelength of 360 to 400 nm and is made of gallium nitride (GaN).

In this embodiment, the control circuit 500 executes continuous control that the switching circuit 400 is controlled so that the light emitting diodes 49m are continuously turned on when the actual voltage of the power source does not exceed the rated voltage of the power source, and executes pulse control that as the actual voltage of the power source exceeds the rated voltage of the power source, time in which the light emitting diodes 49m are turned on is gradually reduced.

Concretely, in this embodiment, the rated voltage of the power source is 13.5 V, when the actual voltage of the power source does not exceed this voltage, continuous control that the switching circuit 400 is controlled so that the light emitting diodes 49m are continuously turned on is executed and when the actual voltage exceeds this voltage, pulse control that as the actual voltage exceeds this voltage, time in which the light emitting diodes 49m are turned on is gradually reduced is executed. When the actual voltage of the power source greatly exceeds a value of the rated voltage of the power source, the light emitting diodes 49m are always turned off to prevent excessive heat.

FIG. 21 shows a signal waveform generated by the triangular wave generator 600. A rectangular pulse P0 (see FIG. 21) is acquired by voltage divided by the resistors 602 and 603 and the resistor 605 in a position A in the triangular wave generator 600. In a position B in the triangular wave generator 600, a triangular pulse P1 (see FIG. 21) is acquired based upon the charge or the discharge of the capacitor 608. The triangular pulse P1 has mean voltage corresponding to the magnitude of the actual voltage of the power source and when the actual voltage of the power source is high, the means voltage of the triangular pulse P1 is also high.

A triangular pulse P1 is input to the noninverting input terminal (+) of the comparator 705 in the comparator 700. Reference voltage Vm is input to the inverting input terminal (−) of the comparator 705 as described above. When a value of the voltage of the triangular pulse P1 is lower than that of the reference voltage Vm, a signal L (Low) is output from the output terminal of the comparator 705, the emitter and the collector of the transistor 401 conduct, the transistor 401 is turned on, the group of light emitting diodes 300 is turned on and emits.

In the meantime, when a value of the voltage of the triangular pulse P1 is higher than that of the reference voltage Vm, a signal H (High) is output from the output terminal of the comparator 705, the emitter and the collector of the transistor 401 do not conduct, the transistor 401 is kept off and the group of light emitting diodes 300 is kept off.

Further, action when the rated voltage of the power source is 13.5 V will be described below. FIG. 22 shows a case that the actual voltage of the power source is equal to or lower than the rated voltage of the power source and is V1 (for example, 9 V). At this time, as shown in FIG. 22, a value of the maximum voltage Vp of a triangular pulse P1 is lower than that of reference voltage Vm. In this case, the transistor 401 which is a switching device is continuously turned on, the light emitting diodes (LED) 49m are continuously turned on and continuously emits.

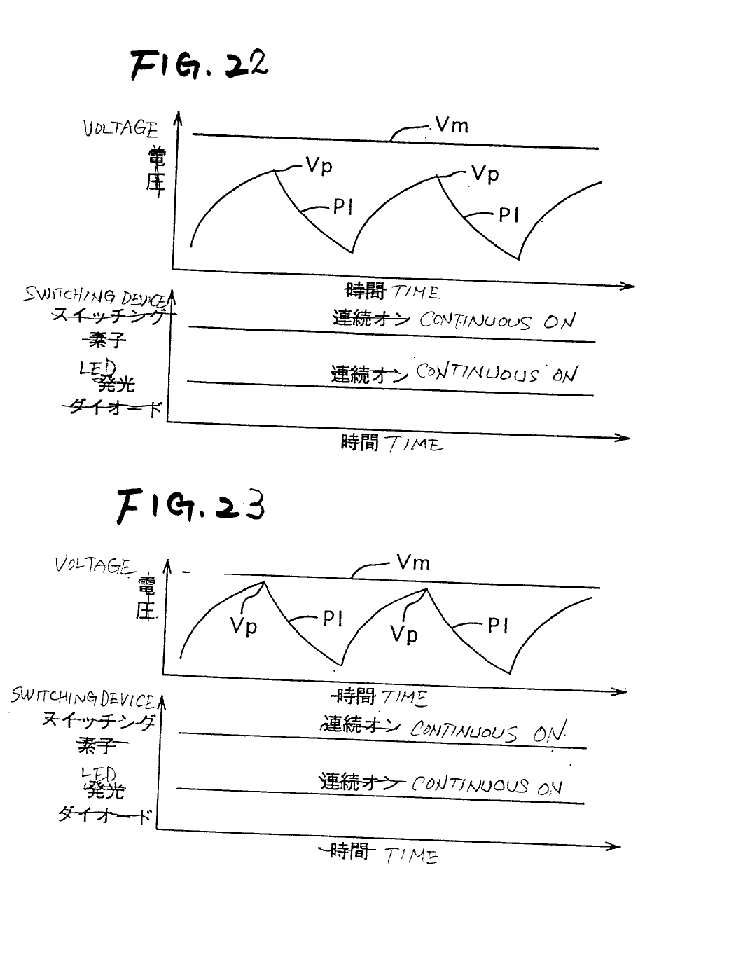

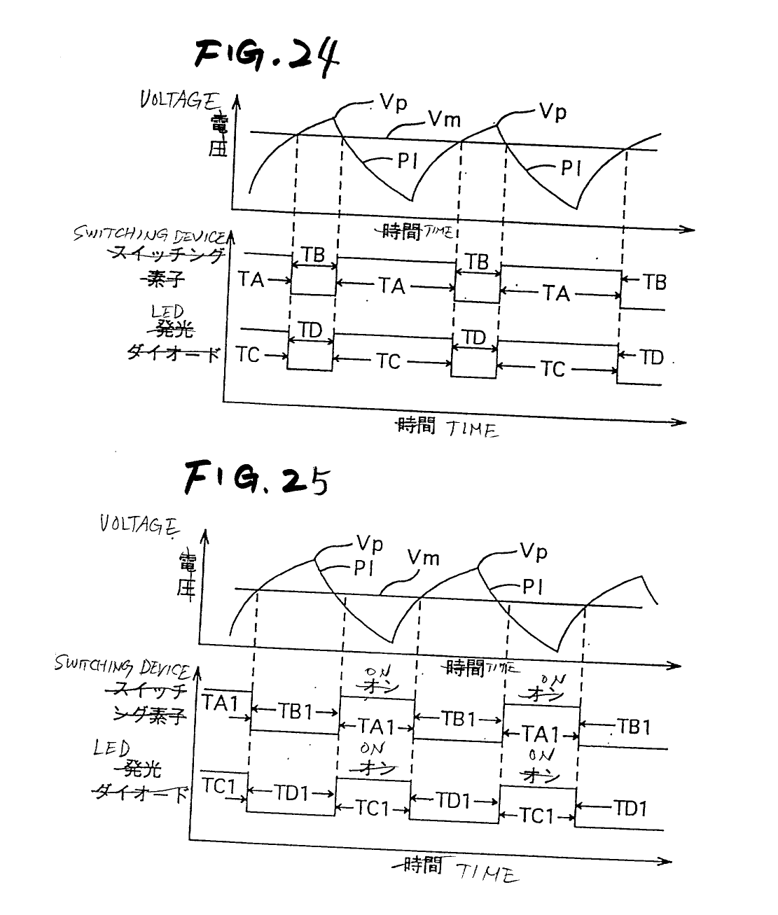

FIG. 23 shows a case that though the actual voltage of the power source is equal to or lower than the rated voltage of the power source, it approaches the rated voltage and is V2 (V2>V1: for example, 13V). At this time, as shown in FIG. 23, a value of the maximum voltage Vp of a triangular pulse P1 is slightly lower than that of reference voltage Vm. In this case, as described above, the transistor 401 which is a switching device is also continuously turned on and the light emitting diodes (LED) 49m also continuously emit.

FIG. 24 shows a case that the actual voltage of the power source exceeds the rated voltage of the power source and is V3 (V3>V2: For example, 14 V). At this time, as shown in FIG. 24, though a value of the maximumvoltage Vp of a triangular pulse P1 exceeds that of reference voltage Vm, a waveform except the vicinity of the maximum voltage Vp does not exceed a value of the reference voltage Vm. In this case, in time TA in which the waveform does not exceed, the transistor 401 which is a switching device is turned on and in time TB in which the waveform exceeds, the transistor 401 which is a switching device is turned off. As a result, pulse control is applied to the light emitting diodes 49m that in time TD in which the value of the maximum voltage VP of the triangular pulse P1 exceeds that of the reference voltage Vm, the light emitting diodes 49m are turned off, and in time TC in which the value does not exceed, the light emitting diodes 49m are turned on. Mean current supplied to the light emitting diodes 49m decreases. In this case, the duty ratio Du of the light emitting diodes 49m is TC/(TC+TD).

FIG. 25 shows a case that the actual voltage of the power source exceeds the rated voltage of the power source and is V4 (V4>V3: for example, 15 V). At this time, as shown in FIG. 25, a value of the maximum voltage Vp of a triangular pulse P1 considerably exceeds that of reference voltage Vm. In this case, in time TA1 in which the actual voltage does not exceed, the transistor 401 which is a switching device is turned on and in time TB1 in which the actual voltage exceeds, the transistor 401 which is a switching device is turned off. As a result, pulse control is applied to the light emitting diodes 49m that in time TD1 in which the value of the maximum voltage VP of the triangular pulse P1 exceeds that of the reference voltage Vm, the light emitting diodes 49m are turned off and in time TC1 in which the value of the maximum voltage does not exceed, the light emitting diodes 49m are turned on. Mean current supplied to the light emitting diodes 49m decreases. In this case, the duty ratio DU of the light emitting diodes 49m is TC1/(TC1+TD1).

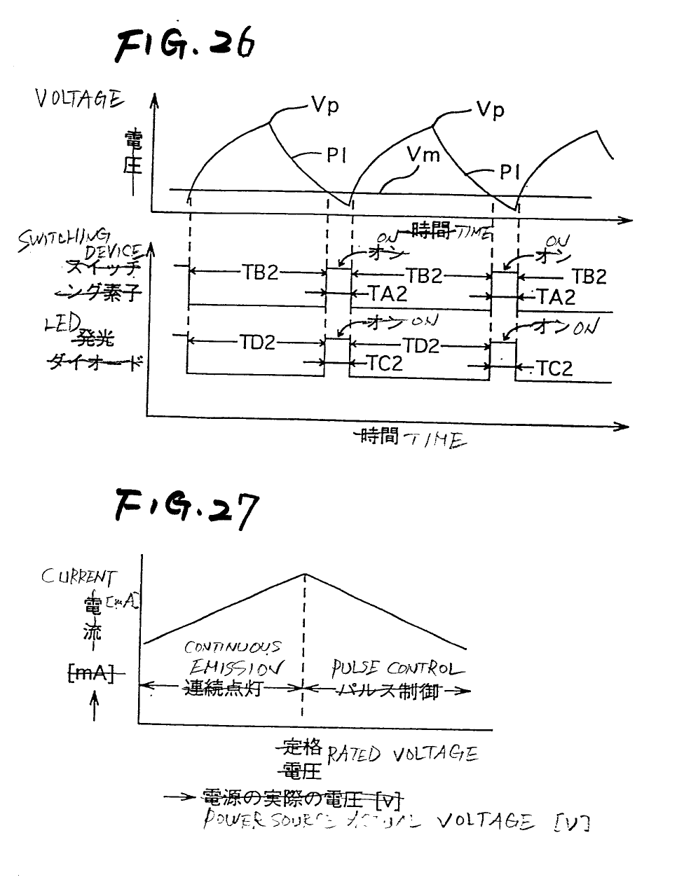

FIG. 26 shows a case that the actual voltage of the power source exceeds the rated voltage of the power source and is V5 (V5>V4: for example, 16 V). At this time, as shown in FIG. 26, a value of the maximum voltage Vp of a triangular pulse P1 is considerably larger than that of reference voltage Vm and most of the waveform of the triangular pulse P1 exceeds the reference voltage Vm. In this case, in time TA2 in which the actual voltage does not exceed, the transistor 401 which is a switching device is turned on and in time TB in which the actual voltage exceeds, the transistor 401 which is a switching device is turned off. As a result, pulse control is applied to the light emitting diodes 49m that in time TD2 in which the value of the maximum voltage Vp of the triangular pulse P1 exceeds that of the reference voltage Vm, the light emitting diodes 49m are turned off and in time TC2 in which the value of the maximum voltage does not exceed, the light emitting diodes 49m are turned on. Mean current supplied to the light emitting diodes 49m decreases. In this case, the duty ratio Du2 of the light emitting diodes 49m is TC2/(TC2+TD2). When each duty ratio is compared, relation, Du>Du1>Du2 is acquired.

As described above, in this embodiment, when the actual voltage of the power source does not exceed the rated voltage of the power source, continuous control that the switching circuit 400 is controlled so that the light emitting diodes 49m are continuously turned on is executed. When the actual voltage of the power source exceeds the rated voltage of the power source, pulse control is applied to the light emitting diodes 49m, as the actual voltage of the power source exceeds the rated voltage of the power source, time in which the light emitting diodes 49m are turned on is gradually reduced, therefore, the calorific value of the light emitting diodes 49m is lowered, and the deterioration and the failure of the light emitting diodes 49m is inhibited.

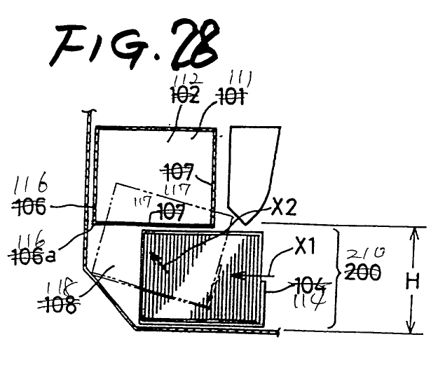

In this embodiment, a characteristic shown in FIG. 27 between the actual voltage of the power source and mean current per unit time fed to the light emitting diodes 49m is acquired. In FIG. 27, the x-axis shows the actual voltage of the power source and the y-axis shows mean current per unit time supplied to the light emitting diodes 49m. While the actual voltage of the power source increases from voltage equal to or lower than the rated voltage to the rated voltage (13.5 V), mean current supplied to the light emitting diodes 49m gradually increases. As the actual voltage of the power source exceeds the rated voltage (for example, 13.5 V), mean current per unit time gradually decreases and when the mean current further decreases, it becomes equal to or lower than its rated value. Particularly, in this embodiment, as understandable from FIG. 27, for the variation of the actual voltage of the power source, the increase and the decrease of mean current linearly vary.

Also, in this embodiment, as the waveform at a leading edge and the waveform at a trailing edge of a signal at a low level and a signal at a high level respectively output from the output terminal of the comparator 705 can be rounded because of the charge and discharge action of the capacitor 707 of the comparator 700, this embodiment can contribute to the reduction of noise and the light emitting diodes 49m can be more securely protected.

A value of the rated voltage of a power source mounted in a vehicle is generally 13.5 V, however, it may considerably vary according to the used situation of electrical equipment of the vehicle. For example, a value of the rated voltage may vary in a range of 9 to 16 V. The activating part 49 equivalent to this embodiment includes the light emitting diode shown in FIG. 1. Therefore, as the light emitting diodes 49m continuously emit light when the actual voltage of the power source of the vehicle does not exceed the rated voltage of the power source and pulse control is applied to the light emitting diodes 49m when the actual voltage of the power source mounted in the vehicle exceeds the rated voltage of the power source, the excess of voltage and the excess of current respectively supplied to the light emitting diodes 49m can be prevented, in addition, the excessive heat of the light emitting diodes 49m is inhibited, the light emitting diodes 49m can be more securely protected and the life of the light emitting diodes 49m can be extended.

In this embodiment in which the light emitting diodes 49m can be more securely protected and the life of the light emitting diodes 49m can be extended even if the voltage of a power source of a vehicle excessively increases as described above, the normal used situation of the light emitting diodes 49m can be set to the vicinity of the rated voltage of the power source. Therefore, in the normal used situation, the light emitting diodes 49m can emit in a full or substantially full state, it is advantageous to activate the photocatalytic holder 4 and the efficiency of cleaning by the air cleaner can be enhanced.

When the actual voltage of a power source of a vehicle is considerably lower (for example, 9 to 10 V) than the rated voltage of the power source, the luminous energy of the light emitting diodes 49m is short, however, as it is only at the starting of the vehicle that a situation that the actual voltage of the power source is considerably lower than the rated voltage of the power source as described above occurs, it is extremely short and temporary, the situation has no effect upon the activation of a photocatalytic substance of the photocatalytic holder 4.

It is desirable to enhance the cleaning performance of the photocatalytic holder 4 that the photocatalytic holder 4 is opposed to the light emitting diodes 49m for activating the photocatalytic holder in as large area as possible and area irradiated by light emitted from the light emitting diodes 49m is increased. At this point, in this embodiment, as the flat photocatalytic holder 4 is used and the side having larger area is directly opposite to the light emitting diodes 49m, the photocatalytic particle 40 can be effectively activated and this embodiment is advantageous to enhance the cleaning performance of the photocatalytic holder 4.

In the above-mentioned embodiment, the first fitting part lm and the second fitting part 51s are both provided to the suction grille 51 of the body 1, however, the invention is not limited to this and in short, they have only to be provided on the side of the body 1. In the above-mentioned embodiment, the photocatalyst holder 4 is held by the blowout grille 54, however, the invention is not limited to this and the filter for collecting dust may be also held or nothing may be also held by the blowout grille 54. In the above-mentioned embodiment, the invention is applied to the blowout grille 54, however, the invention can be also applied to the suction grille though it is not shown. The invention is not limited to only the above-mentioned embodiment shown in the drawings and can be suitably changed as long as the change does not deviate from the object. The above-mentioned embodiment shows the concept of the invention and the word, the member and the word describing the member in the embodiment can be described in each claim even if it is a part.

The following technical concept will be also able to be grasped based upon the above description.

An air cleaner characterized in each claim in that the fitting end face of a first fitting part and the fitting end face of a second fitting part are apart in a direction in which a blowout grille is extended, the fitting end face of the second fitting part is arranged closer to the blowout grille than the fitting end face of the first fitting part and the fitting end face of the first fitting part is arranged farther from the blowout grille than the fitting end face of the second fitting part.

An air cleaner characterized in each claim in that a grille attached to the body of the air cleaner is tilted so that the grille approaches an attachment portion (the ceiling) from a turning arm to a grille fitting part.

An air cleaner characterized in a fourth claim in that at least one of a photocatalyst holder and a grille is provided with inside and outside reverse attachment preventing means for preventing the attachment to the grille when the photocatalyst holder is reversed.

An air cleaner characterized in a first claim in that at least one of a blowout grille and a suction grille is provided.

According to the invention, the body of the air cleaner has the first fitting part and the second fitting part mutually close and opposite via space, when the turned angle of the grille with the body of the air cleaner is small, the turning arm of the grille can be inserted or detached into/from the space between the first fitting part and the second fitting part, when the turned angle of the grille with the body of the air cleaner is large, the turning arm of the grille is fitted to the second fitting part, a part on the back of the surface fitted to the second fitting part out of the turning arm of the grille is fitted to the first fitting part and the grille is temporarily held in the body of the air cleaner. Therefore, in case the blowout grille is detached from the body of the air cleaner, it can be pulled out from the body of the air cleaner if the turned angle of the grille with the body of the air cleaner is reduced and the grille is pulled out in a state in which the turning arm of the grille can be inserted or detached into/from the space between the first fitting part and the second fitting part. Such operation can be easily performed with an operator's one hand and operability is enhanced.

As described above, in this embodiment, as shown in FIG. 8, the mesh members 42 and 43 respectively attached to the ventilation windows 41x and 46x of the frames 41 and 46 forming the photocatalytic holder 4 are formed by braiding thin wire 42a and 43a and have flexibility. Therefore, both the mesh members 42 and 43 can be deflected in a direction in which the photocatalytic particles 40 are stuffed in the housing 4t densely. As described above, multiple photocatalytic particles 40 can be stuffed in the housing 4t densely without rattle utilizing the flexibility of the mesh members 42 and 43 and the mesh members 42 and 43 can directly or indirectly press the photocatalytic particles 40. Therefore, the rattle of the photocatalytic particles 40 in the housing 4t is inhibited and noise caused by the rattle of the photocatalytic particles 40 is also inhibited. As the above-mentioned mesh members 42 and 43 respectively have a mesh opening 42f, 43f in smaller size than the outside diameter of the photocatalytic particle 40, the photocatalytic particle 40 stuffed in the housing 4t is prevented from falling from the mesh member 42 or 43.

Also, in this embodiment, as described above, the elastic member 44 provided with the soft layer 44c touched to the photocatalytic particles 40 is stuffed in the housing 4t together with the photocatalytic particles 40. Therefore, the rattle of the multiple photocatalytic particles 40 stuffed in the housing 4t is more inhibited owing to an elastic deformation function which the elastic member 44 has, compared with a case that no elastic member 44 is provided. Therefore, the damage and the deterioration of the photocatalytic particle 40 caused by friction due to the rattle are more prevented, the durability of the photocatalytic particle 40 is more enhanced and the life is extended. Even if impact and others act upon the photocatalytic holder 4 as when the photocatalytic holder 4 falls on a floor, the elastic member 44 having the soft layer 44c can produce impact softening effect and the photocatalytic particle 40 can be securely protected.

To enhance the cleaning performance of the photocatalytic holder 4, it is desirable that the ventilation of the photocatalytic holder 4 is satisfactorily secured and the transmission of light radiated from the light emitting diodes 49m is satisfactorily secured. At this point, in this embodiment, as the mesh members 42 and 43 respectively have multiple mesh openings 42f and 43f, the ventilation of the photocatalytic holder 4 is secured. Further, the transmission of light radiated from the light emitting diodes 49m is satisfactorily secured. As the elastic member 44 also has multiple mesh openings 44f, the ventilation of the photocatalytic holder 4 is secured and the elastic member is advantageous to enhance cleaning performance. As the photocatalytic holder 4 is flat and is thin in a direction of ventilation, the ventilation of the photocatalytic holder 4 is satisfactorily secured even if the photocatalytic particles 40 are stuffed in the photocatalytic holder 4 and it is advantageous to enhance the cleaning performance of the photocatalytic holder 4 that the photocatalytic holder is flat and thin.

To enhance the cleaning performance of the photocatalytic holder 4, it is desirable that the photocatalytic holder 4 is opposed to the light emitting diodes 49m that activate the photocatalytic holder in as large area as possible and area in which light from the light emitting diodes 49m is received is increased. At this point, in this embodiment, as the photocatalytic holder 4 is made thin and flat and the side having larger area is directly opposed to the light emitting diodes 49m, the photocatalytic particle 40 can be effectively activated and the above-mentioned is advantageous to enhance the cleaning performance of the photocatalytic holder 4.

In the air cleaner equivalent to the above-mentioned embodiment, both the filter 3 for collecting dust and the photocatalytic holder 4 are mounted, however, depending upon a type, though the photocatalytic holder 4 is mounted, the filter 3 for collecting dust may be also omitted.

In the above-mentioned embodiment, the photocatalytic holder 4 is mounted in the air cleaner mounted in a vehicle, however, the invention is not limited to this and the photocatalytic holder can be also mounted in another equipment for which the reduction of pollutant is requested. The outside shape of the photocatalytic holder 4 is not limited to the shape of a thin rectangular box and may be also a circular box. The shape of the arm 41r is not limited to that in the above-mentioned embodiment. In addition, the invention is not limited to only the above-mentioned embodiment shown in the drawings and may be suitably changed in a range which does not deviate from the object. The above-mentioned embodiment shows the concept of the invention, and the word, the member and the word modifying the member respectively described in the embodiment can be described in each claim even in a part.

The following technical concept will be also able to be grasped based upon the above description.

An air cleaner based upon an air cleaner provided with a suction port and a blowout port, an air passage connecting both, a fan for forming air flow from the suction port to the blowout port via the air passage and a cleaning part arranged in the air passage for cleaning air flow flowing in the air passage and characterized in that the cleaning part is provided with a flat photocatalytic holder and flat exciting means arranged along the photocatalytic holder so that the flat exciting means is opposite to the photocatalytic holder. It is advantageous to thin the air cleaner.

An air cleaner characterized in that the photocatalytic holder is flat and is attached to the back of a blowout grille so that the photocatalytic holder can be detached.

A photocatalytic holder or an air cleaner characterized in each claim in that excessive deflection preventing means (an arm 41r) for inhibiting the excessive deflection of a mesh member is provided. The excessive deflection preventing means is advantageous to prevent the damage of the mesh member.

A photocatalytic holder or an air cleaner characterized in a second claim in that an elastic member is arranged on the side far from the exciting means such as a light emitting diode. The deterioration of the elastic member is inhibited.

A photocatalytic holder or an air cleaner characterized in each claim in that so that particles of a photocatalytic substance are stuffed in the photocatalytic holder so that the number of particles arranged along the surface of the photocatalytic holder is many and the number of particles arranged in a direction of the thickness of the photocatalytic holder is small.

According to the photocatalytic holder according to the invention and the air cleaner according to the invention, as the mesh member attached to the ventilation window of a frame forming the photocatalytic holder is flexible, particles of the photocatalytic substance can be deformed in a direction in which they are stuffed in the housing densely. As described above, the particles of the photocatalytic substance can be stuffed in the housing densely utilizing the deflection of the mesh member, the rattle of the particles is inhibited and noise caused by the rattle of the particles of the photocatalytic substance is also inhibited. As the rattle of the particles of the photocatalytic substance is inhibited as described above, the mesh member is advantageous to prevent the damage and the deterioration of the particles caused by friction caused due to the rattle, the durability of particles of the photocatalytic substance can be enhanced and the life can be extended. Particularly as the rattle of the particles is more inhibited in case the elastic member is stuffed in the housing together with the particles, the elastic member is advantageous to further prevent the damage and the deterioration of the particles caused by friction caused due to the rattle. The air cleaner according to the invention is suitable in case it is mounted in a vehicle on which vibration acts in traveling.

As described above, in this embodiment, the filter 3 is inserted or detached by moving the filter 3 in a direction in which the air passage 1c is extended (from the upstream side to the downstream side of air flow or from the downstream side to the upstream side of air flow) from the blowout port 55 which is a filter port, turning the filter 3 utilizing the concave portion 15 provided on the opposite side of the blowout port 55 which is the filter port. As the filter is moved in the direction in which the air passage 1c where air flows is extended and can be inserted or detached as described above, the air passage 1c and a filter insertion path can be made common. Therefore, differently from the prior art shown in FIG. 6, a dedicated filter insertion path for inserting or detaching the filter 3 is not required to be independently formed separately from the air passage 1c. Therefore, the invention can contribute to the downsizing of the body 1 and is suitable for an air cleaner attached to the ceiling of the interior R of a vehicle.

In the above-mentioned embodiment, the filter 3 is formed by weaving thin flexible filter material that passes air in the shape of pleats so that a peak and a trough are alternately formed, however, the invention is not limited to this and mesh material may be also used. The mesh material may also have single-layer structure or plural-layer structure. The whole appearance of the filter 3 is substantially in the shape of a rectangular box, however, the invention is not limited to this and the whole appearance may be also in the shape of a cylinder. Filter material forming the filter 3 has a lamination composed of a layer mainly made of activated carbon and a layer to which an electrification process is applied, however, the invention is not limited to this and the filter material may also have single-layer structure and plural-layer structure. The filter 3 has cushion material 31, however, if sealing performance is kept, the cushion material 31 may be also omitted. The tab 33 that can function as a filter pulling member is integrated with the filter 3, however, if pulling operability is kept, the tab maybe also omitted. In addition, the invention is not limited to only the above-mentioned embodiment shown in the drawings and may be also suitably changed in a range that does not deviate from the object. The above-mentioned embodiment shows the concept of the invention, and the word, the member and the word modifying the member respectively described in the embodiment can be described in each claim even in a part.

The following technical concept will be also able to be grasped based upon the above description.

An air cleaner characterized in each claim in that a filter is in the shape of a box or a cylinder extended in a direction in which an air passage is extended.

An air cleaner characterized in each claim in that a photocatalyst is provided separately from a filter or is integrated with the filter and exciting means for exciting the photocatalyst is arranged in a position in which the exciting means does not interfere with the filter inserted or detached into/from a filter housing.

An air cleaner characterized in each claim in that a filter is formed by flexible filter material.

An air cleaner characterized in each claim in that a filter fitting part for fitting a filter to a filter housing is provided at at least one end of the upstream end and the downstream end of the filter housing.

An air cleaner characterized in each claim in that a filter port is formed on the downstream side of an air passage and a concave portion is formed on the upside of a filter housing.

An air cleaner characterized in each claim in that a wall on which a concave portion is formed is extended along a center line of a filter housing.

An air cleaner characterized in each claim in that the length of a concave portion is slightly shorter than that of a filter.

An air cleaner characterized in each claim in that when a filter is housed in a filter housing, the upstream end of a concave portion is sealed by the upstream end of the filter or the vicinity of the upstream end.

An air cleaner characterized in each claim in that when a filter is housed in a filter housing, the downstream end of a concave portion is sealed by the downstream end of the filter or the vicinity of the downstream end.

An air cleaner characterized in each claim in that a photocatalyst and exciting means for exciting the photocatalyst are arranged on the downstream side of a filter housing in an air passage.

An air cleaner characterized in each claim in that a photocatalyst and exciting means for exciting the photocatalyst are arranged on the downstream side of a filter housing in an air passage and a tilt guide face for guiding a filter so that the upstream end of a filter is upward and the downstream end of the filter is downward is provided between the exciting means and the filter housing.

According to the invention, as the filter is inserted or detached in a direction in which the air passage is extended from the filter port, turning the filter using the concave portion, the air passage and the filter insertion path can be made common. Therefore, differently from the prior art, the dedicated filter insertion path for inserting the filter is not required to be separately provided and the invention can contribute to the downsizing of the air cleaner.

In the above-mentioned embodiment, the inserting pawl member 80 is made wide, however, the invention is not limited to this and it may be also narrow or may be also a bar. One inserting pawl member 80 is provided, however, the invention is not limited to this and two inserting pawl members may be also provided. The two pushing pawl members 90 are provided, however, one or three pushing pawl members may be also provided. The decorative wall is formed by the ceiling wall 200 having a lamination including facing, however, the invention is not limited to this and the decorative wall may also have single-layer structure. In the above-mentioned embodiment, the two regularly fixing parts 13 are provided to the body 1, however, the invention is not limited to this and depending upon a case, one, three, four or more regularly fixing parts may be also provided. The attachment portion is not limited to the reinforcement part which is the reinforcing member of a car body and in short, the attachment portion has only to have strength in which the body 1 of the air cleaner can be attached. In the above-mentioned embodiment, for an absorbing part, both the filter 3 for collecting dust and the photocatalyst 40 are provided, however, the invention is not limited to these and either of the filter 3 for collecting dust or the photocatalyst 40 has only to be provided.

In addition, the invention is not limited to only the above-mentioned embodiment shown in the drawings and can be suitably changed in a range that does not deviate from the object. The above-mentioned embodiment shows the concept of the invention, and the word, the member and the word modifying the member respectively described in the embodiment can be described in each claim even in a part.

The following technical concept will be also able to be grasped based upon the above description.

The mounting structure of an air cleaner based upon mounting structure in which the air cleaner provided with the body having a suction port and a blowout port and having an air passage between both, a fan arranged in the body for forming air flow from the suction port to the blowout port in the air passage and an absorbing part arranged in the air passage is mounted on an attachment portion and characterized in that the body is provided with an inserting pawl member protruded from the end of the body and inserted into an inserting hole of the attachment portion, a pushing pawl member pushed into a pushing hole of an attachment portion in a direction that crosses a direction in which the inserting pawl member is inserted and a regularly fixing part regularly fixed to an attachment portion, the inserting pawl member of the body is inserted into the inserting hole of the attachment portion, the body is temporarily held in the attachment portions by pushing the pushing pawl member of the body into the pushing hole of the attachment portion in the direction that crosses the direction in which the inserting pawl member is inserted and the regularly fixing part of the body is regularly fixed to the attachment portion after temporary holding.

An air cleaner or its mounting structure according to each claim characterized in that an inserting pawl member is wide.

An air cleaner or its mounting structure according to each claim characterized in that the body is in the shape of a thin box and an inserting pawl member is wide in a direction of the width of the body. The inserting pawl member can easily correspond to the thin body.

An air cleaner or its mounting structure according to each claim characterized in that the position of an inserting pawl member can be adjusted in at least a direction (a direction in which the inserting pawl member is inserted) in which the inserting pawl member is protruded in temporary holding. Temporary holding is further facilitated by the adjustment of the position.

An air cleaner or its mounting structure according to each claim characterized in that an inserting pawl member is inserted in a direction in which a ceiling wall is extended in temporary holding. It is suitable for mounting the body on the ceiling.

An air cleaner or its mounting structure according to each claim characterized in that an inserting pawl member and a pushing pawl member are provided at both ends in a direction of the length of the body with their backs to each other.

An air cleaner or its mounting structure according to each claim characterized in that an inserting pawl member has a protruded part on both sides in a direction that crosses an inserted direction.

An air cleaner or its mounting structure according to each claim characterized in that an inserting pawl member is provided at the end in a direction of the length of the body and a pushing pawl member is provided at the back end in the direction of the length of the body.

An air cleaner or its mounting structure according to each claim characterized in that a pushing pawl member of the body can be lifted or lowered in a direction of the height of the body (a direction that crosses a direction in which an inserting pawl member is inserted) in a state in which the inserting pawl member is inserted into an inserting hold of an attachment portion.

An air cleaner or its mounting structure according to each claim characterized in that an attachment portion has an inserting hole into which an inserting pawl member is inserted and the inserting hole is pierced in a direction in which the inserting pawl member is protruded from the body (a direction in which the inserting pawl member is inserted).

Air cleaning structure according to each claim characterized in that a pushing hole of an attachment portion is open in a pierced state in a direction of the height of the body (a direction that crosses a direction in which an inserting pawl member is inserted).

An air cleaner or its mounting structure according to each claim characterized in that the body is supported at three points in temporary held state.

An air cleaner or its mounting structure according to each claim characterized in that a pushing pawl member is made of elastic material such as resin.

An air cleaner or its mounting structure according to each claim characterized in that the position of a pushing pawl member can be adjusted in temporary holding. Temporary holding is further facilitated by the adjustment of the position.

An air cleaner or its mounting structure according to each claim characterized in that the outer end of a grille is curved so that it is protruded toward the ceiling (upward) to enhance performance for biting a decorative wall.

An air cleaner or its mounting structure according to each claim characterized in that the terminal of a decorative wall recedes in a bitten direction because the outer end of a grille bites the terminal of the decorative wall.

According to the invention, the body of the air cleaner is temporarily held in the attachment portions by first inserting the inserting pawl member of the body of the air cleaner into the inserting hold of the attachment portion and pushing the pushing pawl member of the body of the air cleaner into the pushing hole of the attachment portion. After temporary holding, the regularly fixing part of the body of the air cleaner can be regularly fixed to the attachment portion. As the body of the air cleaner is regularly fixed after the body of the air cleaner is temporarily held in the attachment portions as described above, the body of the air cleaner is not required to be supported with a hand and an arm in regularly fixing and further, as unnatural posture is not required to be forced upon an operator, not only the workability of attachment can be enhanced but the invention can also contribute to the enhancement of the safety of an operator.

Further according to the invention, when the body of the air cleaner is attached to the attachment portions, the outer end of the grille is made to bite the decorative wall such as the ceiling wall, nipping the decorative wall such as the ceiling wall with the outer end of the grille and the nipping part in a direction of the thickness of the decorative wall. Hereby, the decorative wall such as the ceiling wall can be more securely fixed and the invention is advantageous to prevent the decorative wall such as the ceiling wall from being peeled. Further, as the outer end of the grille bites the decorative wall such as the ceiling wall, difference in a level in a boundary between the decorative wall such as the ceiling wall and the outer end of the grille is easily reduced and the appearance of the boundary is improved.

A value of the rated voltage of a power source is not limited to the above-mentioned values and is suitably changed according to the application of the power source. In the above-mentioned embodiment, the value (13.5 V) of rated voltage itself functions as a threshold as the criterion of continuous control and pulse control, however, the invention is not limited to this and actual voltage has only to be in a rated voltage area meaning the vicinity of rated voltage. The light emitting diode illuminating device and the photocatalytic converter according to the invention are not limited to the air cleaner having the above-mentioned structure, can be also applied to an air cleaner except the air cleaner having the above-mentioned structure and further, can be also applied to another equipment, another device and another facilities. In addition, the invention is not limited to only the above-mentioned embodiment shown in the drawings and applied examples and can be suitably changed in a range that does not deviate from the object. The above-mentioned embodiment and applied examples show the concept of the invention, and the word, the member and the word modifying the member respectively described in the embodiment and the applied examples can be described in each claim even in a part.

The following technical concept will be also able to be grasped based upon the above description.

A light emitting diode illuminating device, a photocatalytic converter or an air cleaner according to each claim characterized in that the light emitting diode is set so that it emits in a full or substantially full state in the rated voltage area of a power source.

A light emitting diode illuminating device, a photocatalytic converter or an air cleaner according to each claim characterized in that a control circuit executes pulse control that time in which the light emitting diode is turned on is reduced when the actual voltage of a power source exceeds a rated voltage area and controls so that the duty ratio of power fed to the light emitting diode is reduced as an amount in which the actual voltage of the power source exceeds the rated voltage area increases.

According to the light emitting diode illuminating device of the invention, the photocatalytic converter according to the invention or the air cleaner according to the invention, when the actual voltage of the power source does not exceed the rated voltage area, continuous control that the switching circuit is controlled so that the light emitting diode is continuously turned on is executed and when the actual voltage of the power source exceeds the rated voltage area, pulse control that time in which the light emitting diode is turned on is reduced is executed. Therefore, even if the actual voltage of the power source exceeds the rated voltage area, the heat of the light emitting diode is inhibited, the durability of the light emitting diode is enhanced and the life is extended. Further, as the switching circuit is controlled SO that the light emitting diode is continuously turned on when the actual voltage of the power source does not exceed the rated voltage area, the luminous energy of the light emitting diode is secured.

Also, as mean current supplied to the light emitting diode can be set so that it is in a maximum current area when the actual voltage of the power source is in the rated voltage area of the power source, the luminous energy of the light emitting diode can be secured in a full or substantially full state in a general used state high in the frequency of use. That is, the luminous energy of the light emitting diode can be secured in a full or substantially full state in a general normal used situation long in the time of use.

Therefore, according to the photocatalytic converter according to the invention, as the photocatalytic substance is activated by the light emitting diode that produces the above-mentioned effect, the light emitting diode is advantageous to activate and photocatalytic efficiency can be enhanced. Also, according to the air cleaner according to the invention, as the photocatalytic substance of the photocatalytic holder is activated by the light emitting diode that produces the above-mentioned effect, the light emitting diode is advantageous to activate the photocatalytic substance, photocatalytic efficiency can be enhanced and the ratio of cleaned air can be enhanced.

What is claimed is:

1. An air cleaner comprising:
a main body having a suction port and a blowout port thereon, provided with an air passage connecting to said suction port and said blowout port, a fan for forming air flow from said suction port to said blowout port via said air passage, a cleaning part for cleaning air flow flowing in said air passage; and
a grille attached to said main body, covering at least one of said suction port and said blowout port;
wherein said main body is further provided with a first fitting part and a second fitting part mutually close and opposite via space and said grille is provided with a turning arm provided at one end thereof which functions as a center of turning of said grille and a grille fitting part provided at another end thereof for detachably fitting to said main body, and
wherein, when a turned angle of said grille with said main body is small, said turning arm is inserted or detached into/from the space between said first fitting part and said second fitting part, and when the turned angle of said grille with said main body is large, said turning arm is fitted at one side surface thereof to said second fitting part and another side surface thereof is fitted to said first fitting part, thereby said grille is held by said main body on a turned position.

2. An air cleaner according to claim 1, wherein said main body includes further a decorative plate provided on a surface of said main body, and said decorative plate has said first fitting part and said second fitting part.

3. An air cleaner according to claim 1, wherein said grille is further provided with a photocatalyst holder holding a photocatalyst.

4. An air cleaner according to claim 3, wherein said photocatalyst holder is detachably mounted on said grille.

5. A photocatalytic filter comprising:
a frame provided with a housing and a ventilation window communicating with said housing;
multiple particles of a photocatalytic substance stuffed in said housing of said frame; and
a mesh member attached to said ventilation window of said frame and having multiple mesh openings for ventilation, said openings in smaller size than said particles;
wherein said mesh member is brought into pressing contact with said particles, thereby preventing rattle of said particles.

6. A photocatalytic filter according to claim 5, wherein an elastic member contacting with said particles is stuffed in said frame together with said particles.

7. A photocatalytic filter according to claim 6, wherein said elastic member is formed in a meshed shape having mesh openings for ventilation.

8. An air cleaner comprising a suction port, a blowout port, an air passage connecting to said suction port and said blowout port, a fan for forming air flow from said suction port to said blowout port via said air passage and a cleaning part arranged in said air passage for cleaning air flow flowing in said air passage,
wherein said cleaning part is provided with a photocatalytic filter according to claim 5.

9. An air cleaner according to claim 8, further comprising:
a grille mounted on at least one of said suction port and said blowout port,
wherein at least one of said photocatalytic filter and said grille is provided with an attachment preventing part for preventing the attachment of said photocatalytic filter to said grille when the inside and the outside of said photocatalytic filter are reversed.

10. An air cleaner comprising:
an air passage extended in said air cleaner;
a filter housing provided on a part of said air passage;
a fan for forming air flow flowing in said air passage; and
a filter accommodated in said filter housing and to which pollutant included in air flow adheres;
a filter port forming a part of the air passage; and
a concave portion communicating with said filter housing, provided on an opposite side to said filter port via a center line in a direction of air flow of said filter housing for promoting a turning of said filter;
wherein said filter is inserted into and detached from said filter housing by moving said filter in a direction in which said air passage is extended from said filter port and by turning said filter using said concave portion.

11. An air cleaner according to claim 10, wherein said filter port is a blowout port on a downstream side of said air passage.

12. An air cleaner according to claim 10, wherein said concave portion has a guide part for assisting the turning of said filter on at least one of an upstream side and the downstream side of said filter housing.

13. An air cleaner comprising:
a main body having a suction port and a blowout port;
an air passage provided between said suction port and blowout port;
a fan arranged in said main body for forming air flow from said suction port to said blowout port in said air passage;
an absorbing part arranged in said air passage;
wherein said main body is provided with an inserting pawl member protruded at an end of said main body and inserted into an inserting hole formed on an attachment portion, a pushing pawl member pushed into a pushing hole formed on said attachment portion in a direction that crosses a direction in which said inserting pawl member is inserted and a regularly fixing part regularly fixed to said attachment portion; and
said main body is held on said attachment portion by inserting said inserting pawl member into said inserting hole and pushing said pushing pawl member into said pushing hole in the direction that crosses the direction in which said inserting pawl member is inserted and after temporary holding, said regularly fixing part is regularly fixed to said attachment portion.

14. An air cleaner according to claim 13, further comprising
a grille forming at least one of said suction port and said blowout port, and
a nipping part for nipping a part of a decorative wall together with said grille,
wherein an outer end of said grille is set so that it bites a part of said decorative wall by nipping.

15. A light emitting diode illuminating device comprising:
a light emitting diode connected to a power source;
a switching circuit for turning on and off said light emitting diode; and
a control circuit controlling said switching circuit;
wherein said control circuit executes continuous control that said switching circuit is controlled so that said light emitting diode is continuously turned on when an actual voltage of said power source does not exceed a rated voltage area of said power source and executes pulse control that time in which said light emitting diode is turned on is reduced when the actual voltage of said power source exceeds the rated voltage area of said power source.

16. A light emitting diode illuminating device according to claim 15,
wherein said control circuit controls so that when a x-axis shows the actual voltage of said power source and a y-axis shows mean current supplied to said light emitting diode, the mean current increases until the actual voltage of said power source reaches the rated voltage area of said power source and the mean current decreases as the actual voltage of said power source exceeds the rated voltage area of said power source and controls so that when the actual voltage of said power source is in the rated voltage area of said power source, mean current per unit time supplied to said light emitting diode is in a maximum current area.

17. A light emitting diode illuminating device according to claim 15,
wherein said control circuit is provided with a triangular wave generator for generating a triangular pulse having mean voltage corresponding to the actual voltage of a power source and a comparator for comparing the voltage of a triangular pulse output from said triangular wave generator and reference voltage; and
said comparator outputs a signal for turning on said light emitting diode to said switching circuit when the voltage of the triangular pulse is lower than the reference voltage; and
said comparator also outputs a signal for turning off said light emitting diode to said switching circuit when the voltage of the triangular pulse is higher than the reference voltage.

18. A photocatalytic converter provided with a photocatalytic substance and an activating part for activating said photocatalytic substance, wherein said activating part is composed of said light emitting diode illuminating device according to claim 15.

19. An air cleaner comprising:
a suction port and a blowout port, an air passage connecting said suction port and said blowout port, a fan for forming air flow from said suction port to said blowout port via said air passage, a photocatalytic filter including a photocatalytic substance arranged in said air passage for cleaning air flow flowing in said air passage and an activating part for activating said photocatalytic substance of said photocatalytic filter, wherein said activating part is composed of a light emitting diode illuminating device according to claim 15.
